(19) United States
(12) Patent Application Publication
(10) Pub. No.: US 2003/0125336 A1
(43) Pub. Date: Jul. 3, 2003

(54) Hydrohalide salts of an HIV protease inhibitor

(76) Inventors: Fred J. Fleitz, Franklin Park, NJ (US); Daniel E. Petrillo, Hoboken, NJ (US); Yaling Wang, Westfield, NJ (US); Russell R. Ferlita, Milltown, NJ (US); David C. Dubost, Collegeville, PA (US); Joseph D. Armstrong III, Westfield, NJ (US)

Correspondence Address: MERCK AND CO INC, P O BOX 2000, RAHWAY, NJ, 070650907

(21) Appl. No.: 10/158,630

(22) Filed: May 30, 2002

Related U.S. Application Data
(60) Provisional application No. 60/294,372, filed on May 30, 2001.

(51) Int. Cl. A61K 31/496; C07D 413/14

(52) U.S. Cl. 514/253.1; 544/360

(57) ABSTRACT

Pharmaceutically acceptable hydrochloride and hydrobromide salts of Compound A are disclosed, wherein Compound A is of formula:

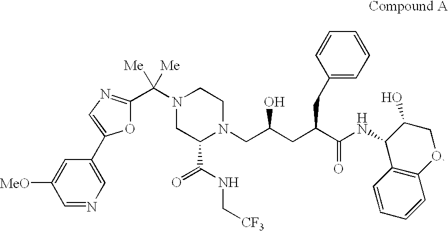

Compound A and its hydrobromide and hydrochloride salts are HIV protease inhibitors useful for preventing or treating HIV infection, for delaying the onset of AIDS, and for treating AIDS. Pharmaceutical compositions employing the crystalline salts, and processes for making and using the crystalline salts are also disclosed.

### FIELD OF THE INVENTION

The present invention is directed to pharmaceutically acceptable hydrohalide salts of an HIV protease inhibitor, Compound A as defined below. The present invention is also directed to processes for preparing hydrohalide salts of Compound A and methods for using the salts.

### BACKGROUND OF THE INVENTION

The HIV retrovirus is the causative agent for AIDS. The HIV-1 retrovirus primarily uses the CD4 receptor (a 58 kDa transmembrane protein) to gain entry into cells, through high-affinity interactions between the viral envelope glycoprotein (gp 120) and a specific region of the CD4 molecule found in T-lymphocytes and CD4 (+) T-helper cells (Lasky L. A. et al., Cell 1987, 50: 975-985). HIV infection is characterized by an asymptomatic period immediately following infection that is devoid of clinical manifestations in the patient. Progressive HIV-induced destruction of the immune system then leads to increased susceptibility to opportunistic infections, which eventually produces a syndrome called AIDS-related complex (ARC) characterized by symptoms such as persistent generalized lymphadenopathy, fever, and weight loss, followed itself by full blown AIDS.

As in the case of several other retroviruses, HIV encodes the production of a protease which carries out post-translational cleavage of precursor polypeptides in a process necessary for the formation of infectious virions (S. Crawford et al., J. Virol. 1985, 53: 899). These gene products include pol—which encodes the virion RNA-dependent DNA polymerase (reverse transcriptase), an endonuclease, and HIV protease—and gag—which encodes the core-proteins of the virion. (H. Toh et al., EMBO J. 1985, 4: 1267; L. H. Pearl et al., Nature 1987, 329-351; M. D. Power et al., Science 1986, 231: 1567).

A number of synthetic anti-viral agents targeted to various stages in the replication cycle of HIV have been disclosed. These agents include inhibitors of HIV cellular fusion (Turpin et al., Expert Opinion on Therapeutic Patents 2000, 10: 1899-1909), reverse transcriptase inhibitors (e.g., didanosine, zidovudine (AZT), and efavirenz), integrase inhibitors (Neamati, Expert Opinion on Investigational Drugs 2000, 10: 281-296), and protease inhibitors (e.g., indinavir, ritonavir, and saquinavir). Protease inhibitors inhibit the formation of infectious virions by interfering with the processing of viral polyprotein precursors. Processing of these precursor proteins requires the action of virus-encoded proteases which are essential for replication (Kohl, N. E. et al., Proc. Natl. Acad. Sci. USA 1988, 85: 4686).

A substantial and persistent problem in the treatment of AIDS has been the ability of the HIV virus to develop resistance to the therapeutic agents employed to treat the disease. Resistance to HIV-1 protease inhibitors has been associated with 25 or more amino acid substitutions in both the protease and the cleavage sites. Many of these viral variants are resistant to all of the HIV protease inhibitors currently in clinical use. See Condra et al., Drug Resistance Updates 1998, 1: 1-7; Condra et al., Nature 1995, 374: 569-571; Condra et al., J. Virol. 1996, 70: 8270-8276; Patrick et al., Antiviral Ther. 1996, Suppl. 1: 17-18; and Tisdale et al., Antimicrob. Agents Chemother. 1995, 39: 1704-1710.

The compound (αR,γS,2S)-N-[(3S,4S)-3,4-dihydro-3-hydroxy-2H-1-benzopyran-4-yl]-y-hydroxy-4-[1-[5-(5-methoxy-3-pyridinyl)-2-oxazolyl]-1-methylethyl]-α-(phenylmethyl)-2-[[(2,2,2-trifluoroethyl)amino]carbonyl]-1-piperazinepentanamide (hereinafter designated herein as “Compound A”) is an HIV protease inhibitor which is much more potent against HIV viral mutants than protease inhibitors presently in clinical use. The structure of Compound A is as follows:

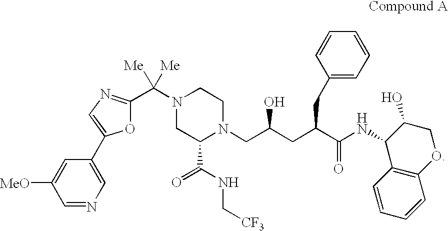

Compound A free base has relatively low solubility in aqueous solutions including dilute HCl (gastric acid) solutions and concomitantly has exhibited poor oral absorption in animal models. Preparation of an acceptable salt of Compound A suitable for pharmaceutical development proved problematic. Numerous attempts to isolate a crystalline salt form of Compound A have failed. Accordingly, there has been a need to develop a pharmaceutically acceptable salt of Compound A which has sufficient solubility in aqueous environments and has relatively good oral bioavailability and a good pharmacokinetic profile.

### SUMMARY OF THE INVENTION

The present invention is directed to pharmaceutically acceptable hydrohalide salts of an HIV protease inhibitor. More particularly, the present invention includes a hydrohalide salt of Compound A, wherein the hydrohalide is hydrochloride or hydrobromide. Embodiments of the present invention include crystalline hydrochloride salts of Compound A and a crystalline hydrobromide salt of Compound A.

The present invention also includes processes for preparing the hydrochloride and hydrobromide salts of Compound A and methods of using the Compound A salts for inhibiting HIV protease, for preventing or treating HIV infection, and for treating or delaying the onset of AIDS.

The crystalline HCl and HBr salts of Compound A of the present invention have relatively high solubility in aqueous solutions including dilute HCl (gastric acid) solutions relative to Compound A per se. The salts also exhibit superior oral absorption and improved pharmacokinetics in animal models compared to Compound A per se.

Other salt forms of Compound A have been prepared (e.g., sulfate, benzenesulfonate, and methanesulfonate salts), but only as amorphous materials or oils. Oils and amorphous salts are generally not suitable for pharmaceutical use, because their properties are typically difficult to quantify and change with time, which can adversely and unpredictably affect the safety and efficacy of a dosage regimen. By contrast, the hydrochloride and hydrobromide salts of Compound A of the present invention can be prepared in crystalline form and thus are more suitable for pharmaceutical use than the other salt forms.

The foregoing embodiments and other embodiments, aspects and features of the present invention are either further described in or will be apparent from the ensuing description, examples, and appended claims.

### DETAILED DESCRIPTION OF THE INVENTION

The present invention provides pharmaceutically acceptable hydrohalide salts of the potent HIV protease inhibitor, Compound A, pharmaceutical compositions containing the salts, and methods of making and using the hydrohalide salts of Compound A. The Compound A hydrohalide salts and pharmaceutical compositions of the present invention are useful for inhibiting HIV protease, preventing infection by HIV, treating infection by HIV, delaying the onset of AIDS, and treating AIDS, in adults, children or infants. Delaying the onset of AIDS, treating AIDS, or preventing or treating infection by HIV is defined as including, but not limited to, treating a wide range of states of HIV infection: AIDS, ARC, both symptomatic and asymptomatic, and actual or potential exposure to HIV. For example, the salts and pharmaceutical compositions of this invention are useful in treating infection by HIV after suspected past exposure to HIV by, e.g., blood transfusion, exchange of body fluids, bites, accidental needle stick, or exposure to patient blood during surgery. The salts of the invention can also be used in “salvage” therapy; i.e., the salts of Compound A can be used to treat HIV infection, AIDS, or ARC in HIV-positive subjects whose viral load achieved undetectable levels via conventional therapies employing known protease inhibitors, and then rebounded due to the emergence of HIV mutants resistant to the known inhibitors.

The term “Compound A,” as used herein refers to the free base of formula:

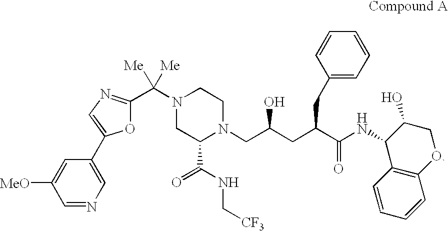

The term “hydrohalide salt” refers to any hydrochloride or hydrobromide salt of Compound A.

Compound A and its hydrohalide salts are inhibitors of HIV protease, including mutant forms thereof that are resistant to known protease inhibitors. Compound A has exhibited IC₅₀ values below 1 nM against the wild-type enzyme and below 5 nM against the mutant enzymes Q-60, K-60, and V-18 in the assay for inhibition of microbial expressed HIV protease described in International Publication No. WO 01/05230. Compound A has also exhibited CIC₉₅ values below 50 nM against the wild-type viral construct and CIC₉₅ values below 125 nM against the viral constructs Q60, K-60, and V-18 in the cell spread assay described in WO 01/05230. Compound A is significantly more potent in both of these assays than indinavir.

An embodiment of the present invention is a crystalline hydrochloride salt of Compound A, which is a Form I crystalline hydrochloride salt of Compound A, characterized by crystallographic d-spacings of 4.2, 4.9, and 5.7 angstroms. Another embodiment of the present invention is the Form I crystalline hydrochloride salt of Compound A characterized by crystallographic d-spacings of 3.8, 3.9, 4.2, 4.3, 4.9, and 5.7 angstroms. Still another embodiment of the present invention is the Form I crystalline hydrochloride salt of Compound A characterized by crystallographic d-spacings of 3.1, 3.5, 3.8, 3.9, 4.2, 4.3, 4.9, 5.7, 6.6 and 15.7 angstroms. In an aspect of each of the three preceding embodiments, the Form I crystalline hydrochloride salt is further characterized by a differential scanning calorimetry curve, at a heating rate of 10° C./min in a closed cup under flowing nitrogen, exhibiting an endotherm with a peak temperature of about 207° C. and an associated heat of about 132 J/gm.

Another embodiment of the present invention is a crystalline hydrochloride salt of Compound A, which is a Form II crystalline hydrochloride salt of Compound A, characterized by crystallographic d-spacings of 5.0, 6.4, and 15.7 angstroms. Another embodiment of the present invention is the Form II crystalline hydrochloride salt of Compound A characterized by crystallographic d-spacings of 4.5, 5.0, 5.6, 6.2, 6.4, and 15.7 angstroms. Still another embodiment of the present invention is the Form II crystalline hydrochloride salt of Compound A characterized by crystallographic d-spacings of 3.7, 3.9, 4.5, 4.6, 5.0, 5.6, 6.2, 6.4, 6.7 and 15.7 angstroms. In an aspect of each of the three preceding embodiments, the Form II crystalline hydrochloride salt is further characterized by a differential scanning calorimetry curve, at a heating rate of 10° C./min in a closed cup under flowing nitrogen, exhibiting an endotherm with a peak temperature of about 174.8° C. and an associated heat of fusion of about 48.6 J/gm.

Still another embodiment of the present invention is a crystalline hydrobromide salt of Compound A, which is characterized by crystallographic d-spacings of 3.8, 3.9, and 4.2 angstroms. Another embodiment of the present invention is the crystalline hydrobromide salt of Compound A characterized by crystallographic d-spacings of 3.1, 3.8, 3.9, 4.2, 4.8, and 5.6 angstroms. Still another embodiment of the present invention is the crystalline hydrobromide salt of Compound A characterized by crystallographic d-spacings of 3.1, 3.8, 3.9, 4.2, 4.8, 5.6, 6.7, 10.4 and 15.8 angstroms. In an aspect of each of the three preceding embodiments, the crystalline hydrobromide salt is further characterized by a differential scanning calorimetry curve, at a heating rate of 10° C./min in a closed cup under flowing nitrogen, exhibiting an endotherm with a peak temperature of about 204° C. and an associated heat of about 78 J/gm.

The crystallographic d-spacings set forth in the foregoing embodiments can be determined from the XRPD pattern of the particular Compound A salt.

The present invention includes pharmaceutical compositions comprising a hydrohalide salt of Compound A as originally defined above or as set forth in any of the foregoing embodiments or aspects and a pharmaceutically acceptable carrier.

The present invention also includes pharmaceutical compositions made by combining a hydrohalide salt of Compound A as originally defined above or as set forth in any of the foregoing embodiments or aspects and a pharmaceutically acceptable carrier.

Other embodiments of the present invention include the following:

(a) A method of preventing or treating HIV infection, which comprises administering a therapeutically effective amount of a hydrohalide salt of Compound A.

(b) A method of delaying the onset of AIDS, which comprises administering a therapeutically effective amount of a hydrohalide salt of Compound A.

(c) A method of treating AIDS, which comprises administering a therapeutically effective amount of a hydrohalide salt of Compound A.

(d) A method of inhibiting HIV protease, which comprises administering a therapeutically effective amount of a hydrohalide salt of Compound A.

(e) A method of preventing or treating HIV infection, which comprises administering a pharmaceutical composition comprising a therapeutically effective amount of a hydrohalide salt of Compound A and a pharmaceutically acceptable carrier.

(f) A method of delaying the onset of AIDS, which comprises administering a pharmaceutical composition comprising a therapeutically effective amount of a hydrohalide salt of Compound A and a pharmaceutically acceptable carrier.

(g) A method of treating AIDS, which comprises administering a pharmaceutical composition comprising a therapeutically effective amount of a hydrohalide salt of Compound A and a pharmaceutically acceptable carrier.

(h) A method of inhibiting HIV protease, which comprises administering a pharmaceutical composition comprising a therapeutically effective amount of a hydrohalide salt of Compound A and a pharmaceutically acceptable carrier.

(i) The method of (a) or (b) or (c) or (d), wherein the hydrohalide salt of Compound A is administered in combination with a therapeutically effective amount of at least one AIDS treatment agent selected from the group consisting of AIDS antiviral agents, immunomodulators, and anti-infective agents.

(j) The method of (a) or (b) or (c) or (d), wherein the hydrohalide salt of Compound A is administered in combination with a therapeutically effective amount of at least one antiviral agent selected from the group consisting of non-nucleoside HIV reverse transcriptase inhibitors and nucleoside HIV reverse transcriptase inhibitors.

(k) The method of (a) or (b) or (c) or (d), wherein the hydrohalide salt of Compound A is administered in combination with a cytochrome P450 monooxygenase inhibitor in an amount effective to improve the pharmacokinetics of Compound A.

Additional embodiments of the invention include the methods set forth in (a)-(k) above, wherein the hydrohalide salt of Compound A employed therein is a Compound A hydrohalide salt as set forth in any one of the embodiments or aspects of the salts as described above.

The present invention also includes a process for preparing a hydrohalide salt of Compound A, which comprises dissolving Compound A free base in a solvent and treating the resulting solution with HCl or HBr to form the hydrohalide salt. Suitable solvents for dissolution of Compound A include C₃-C₁₀ linear and branched alkanes, C₁-C₁₀ linear and branched halogenated alkanes, C₅-C₁₀ cycloalkanes, C₆-C₁₄ aromatic hydrocarbons, dialkyl ethers wherein each alkyl is independently a C₁-C₆ alkyl, C₁-C₆ alkyl alcohols, C₁-C₆ alkanes substituted with two C₁-C₆ alkoxy groups, C₄-C₈ cyclic ethers and diethers, C₆-C₈ aromatic ethers, and C₁-C₆ alkyl esters of C₁-C₆ alkylcarboxylic acids. In one embodiment, the solvent is a C₁-C₆ alkyl ester of C₁-C₆ alkylcarboxylic acid. In an aspect of the preceding embodiment, the solvent is a C₁-C₄ alkyl acetate. In another aspect of the preceding embodiment, the solvent is IPAc.

The solution of Compound A free base can be treated with HX directly (X=Cl or Br) such as by bubbling gaseous HX into the solution, or HX can first be dissolved in a suitable solvent and then added as a solution of HX. The solution of HX can be added all at once or can be added in two or more portions, or can be added incrementally. Suitable solvents for dissolving HX include those set forth above as suitable for dissolving Compound A. The solvent can be the same as or different from the solvent employed to dissolve Compound A, provided that the choice of HX solvent does not adversely affect the solubility of Compound A when the two solutions are added together during the treatment step.

HX can be added to the Compound A solution in any proportion with respect to Compound A which results in the formation of at least some of the desired HX salt. However, HX is typically added in a proportion which, under the treatment conditions employed (e.g., temperature, degree of agitation), will permit conversion of at least a major portion (and more often substantially all to all) of Compound A to the desired salt. Accordingly, HX is typically added in an amount of from about 0.9 to about 5 equivalents of HX per equivalent of Compound A, and is more typically added in an amount of from about 1 to about 2 equivalents per equivalent of Compound A. In one embodiment, Compound A is dissolved in a solvent and treated with from about 1.0 to about 1.1 equivalents of HX per equivalent of Compound A.

The treatment of the Compound A solution with HX can be conducted at any temperature at which Compound A is soluble in the chosen solvent. Typically, the treatment step is conducted at a temperature in the range of from about 10 to about 80° C., and more typically at a temperature in the range of from about 20 to about 80° C.

Following the addition of HX, the solution can be aged for a period of time to permit intimate mixing of HX and Compound A. As used herein, the term “aging” and variants thereof (e.g., “raged”) mean allowing the reactants (i.e., HX and Compound A) to stay in contact for a time and under conditions effective for completion of the reaction. The Compound A solution is optionally agitated (e.g., stirred) during HX addition and optionally also during any subsequent aging. At the completion of the treatment step, the desired hydrohalide salt can be recovered by filtration, optionally after cooling or concentrating (e.g., by evaporative removal of solvent by the application of heat and/or vacuum) the treated solution.

An embodiment of the process for preparing a hydrohalide salt of Compound A is the process as defined and describe above, wherein the hydrohalide salt is crystalline. In this embodiment, the Compound A solution can optionally also be seeded with Compound A.HX seed crystal(s) before, during or subsequent to the addition of HX to promote crystal formation. In an aspect of this embodiment, the hydrohalide salt is a Form I crystalline hydrochloride salt characterized by crystallographic d-spacings of 4.2, 4.9, and 5.7 angstroms. Another aspect of this embodiment is the Form I crystalline hydrochloride salt of Compound A characterized by crystallographic d-spacings of 3.8, 3.9, 4.2, 4.3, 4.9, and 5.7 angstroms. Still another aspect of this embodiment is the Form I crystalline hydrochloride salt of Compound A characterized by crystallographic d-spacings of 3.1, 3.5, 3.8, 3.9, 4.2, 4.3, 4.9, 5.7, 6.6 and 15.7 angstroms. Other aspects of this embodiment include the Form I crystalline hydrochloride salt as characterized in any of the three preceding aspects, further characterized by a differential scanning calorimetry curve, at a heating rate of 10° C./min in a closed cup under flowing nitrogen, exhibiting an endotherm with a peak temperature of about 207° C. and an associated heat of about 132 J/gm.

In still another aspect of the preceding embodiment, the hydrohalide salt is a crystalline hydrobromide salt characterized by crystallographic d-spacings of 3.8, 3.9, and 4.2 angstroms. Another aspect of this embodiment of the present invention is the crystalline hydrobromide salt of Compound A characterized by crystallographic d-spacings of 3.1, 3.8, 3.9, 4.2, 4.8, and 5.6 angstroms. Still another aspect of this embodiment is the crystalline hydrobromide salt of Compound A characterized by crystallographic d-spacings of 3.1, 3.8, 3.9, 4.2, 4.8, 5.6, 6.7, 10.4 and 15.8 angstroms. Further aspects of this embodiment is the crystalline hydrobromide salt as characterized in any of the three preceding aspect, further characterized by a differential scanning calorimetry curve, at a heating rate of 10° C./min in a closed cup under flowing nitrogen, exhibiting an endotherm with a peak temperature of about 204° C. and an associated heat of about 78 J/gm.

Another embodiment of the process for preparing a hydrohalide salt of Compound A is a process for preparing a crystalline hydrochloride or hydrobromide salt, which comprises:

(A) dissolving Compound A free base in a solvent to form a solution, wherein the solvent comprises a C₃-C₁₀ linear and branched alkane, a C₁-C₁₀ linear and branched halogenated alkane, a C₅-C₁₀ cycloalkane, a C₆-C₁₄ aromatic hydrocarbon, a dialkyl ether wherein each alkyl is independently a C₁-C₆ alkyl, a C₁-C₆ alkyl alcohol, a C₁-C₆ alkane substituted with two C₁-C₆ alkoxy groups, a C₄-C₈ cyclic ether or diether, C₆-C₈ aromatic ether, or a C₁-C₆ alkyl ester of a C₁-C₆ alkylcarboxylic acid; and

(B) treating the solution formed in Step A with HX to form the crystalline hydrohalide salt, wherein HX is HCl or BBr.

Still another embodiment of the process for preparing a hydrohalide salt of Compound A is a process for preparing a crystalline hydrochloride or hydrobromide salt, which comprises:

(A) dissolving Compound A free base in a C₁-C₆ alkyl ester of C₁-C₆ alkylcarboxylic acid; and

(B) treating the solution of Step A with HX dissolved in a C₁-C₆ alkyl alcohol to form the crystalline HX salt.

Still another embodiment of the process for preparing a hydrohalide salt of Compound A is a process for preparing a crystalline hydrochloride or hydrobromide salt, which comprises (A) dissolving Compound A free base in isopropyl acetate; and (B) treating the solution of Step A with HX dissolved in isopropyl alcohol. Isopropyl alcohol is a good solvent of Compound A and also a good anti-solvent for both the HCl salt and the HBr salt. In a feature of this aspect, the IPAc:IPA volume ratio is typically in a range of from about 2:1 to about 1:2, and is more typically about 1:1.

In an aspect of any one of the three preceding embodiments, the hydrohalide salt is a Form I crystalline hydrochloride salt characterized by crystallographic d-spacings of (a) 4.2, 4.9, and 5.7 angstroms, or (b) 3.8, 3.9, 4.2, 4.3, 4.9, and 5.7 angstroms or (c) 3.1, 3.5, 3.8, 3.9, 4.2, 4.3, 4.9, 5.7, 6.6 and 15.7 angstroms; and is optionally further characterized by a differential scanning calorimetry curve, at a heating rate of 10° C./min in a closed cup under flowing nitrogen, exhibiting an endotherm with a peak temperature of about 207° C. and an associated heat of about 132 J/gm. In another aspect of any one of the three preceding embodiments, the hydrohalide salt is a crystalline hydrobromide salt characterized by crystallographic d-spacings of (a) 3.8, 3.9, and 4.2 angstroms, (b) 3.1, 3.8, 3.9, 4.2, 4.8, and 5.6 angstroms, or (c) 3.1, 3.8, 3.9, 4.2, 4.8, 5.6, 6.7, 10.4 and 15.8 angstroms; and is optionally further characterized by a differential scanning calorimetry curve, at a heating rate of 10° C./min in a closed cup under flowing nitrogen, exhibiting an endotherm with a peak temperature of about 204° C. and an associated heat of about 78 J/gm.

The present invention also includes the hydrohalide salt of Compound A prepared by the process as originally defined and described above and the hydrohalide salts prepared by each of the embodiments and aspects thereof.

The present invention also includes a process for preparing a Form II crystalline hydrochloride salt of Compound A which comprises dissolving a hydrochloride salt of Compound A in acetonitrile, and precipitating the Form II crystalline hydrochloride salt from the solution. In one embodiment of the process, the Compound A HCl salt is dissolved in hot acetonitrile and after dissolution is complete, the solution is cooled to precipitate the Form II crystalline salt. In another embodiment of the process, the Compound A HCl salt solution is concentrated (e.g., by evaporation of acetonitrile using a vacuum and/or heat) to cause precipitation of the Form II crystalline salt. In an aspect of this process and the foregoing embodiments, the Compound A HCl salt which is dissolved in acetonitrile is Form I crystalline hydrochloride salt of Compound A.

The present invention also includes the Form II hydrochloride salt of Compound A prepared by the process as originally defined and described in the preceding paragraph and as prepared by each of the embodiments and aspects thereof.

The present invention also includes a process for preparing Form II crystalline hydrochloride salt of Compound A (wherein Form II is defined and described above), which comprises dissolving Compound A free base in acetonitrile and treating the resulting solution with HCl to form the Form II hydrochloride salt. The solution of Compound A free base can be treated with HCl directly such as by bubbling gaseous HCl into the solution, or HCl can first be dissolved in acetonitrile and then added as a solution of HCl. The solution of HCl can be added all at once or can be added in two or more portions, or can be added incrementally. The treatment of the Compound A solution with HCl can be conducted at any temperature at which Compound A is soluble in acetonitrile, but typically the treatment step is conducted at a temperature in the range of from about 20 to about 80° C., and more typically at a temperature in the range of from about 30 to about 80° C. HCl can be added to the Compound A acetonitrile solution in any proportion with respect to Compound A which results in the formation of at least some of the desired HCl salt, but HCl is typically added in an amount of from about 0.9 to about 5 equivalents of HCl per equivalent of Compound A, and is more typically added in an amount of from about 1 to about 2 equivalents per equivalent of Compound A. In one embodiment, Compound A is dissolved in acetonitrile and treated with from about 1.0 to about 1.1 equivalents of HCl per equivalent of Compound A. Following the addition of HCl, the acetonitrile solution can be aged for a period of time to permit intimate mixing of HCl and Compound A.

The present invention also includes the Form II hydrochloride salt of Compound A prepared by the process as originally defined and described in the preceding paragraph and as prepared by each of the embodiments and aspects thereof.

As noted above, the present invention includes pharmaceutical compositions useful for inhibiting HIV protease, comprising an effective amount of a hydrohalide salt of Compound A and a pharmaceutically acceptable carrier. Pharmaceutical compositions useful for preventing or treating infection by HIV, for delaying the onset of AIDS, or for treating AIDS, are also encompassed by the present invention, as well as a method of inhibiting HIV protease, and a method of preventing or treating infection by HIV, or delaying the onset of AIDS, or treating AIDS. An aspect of the present invention is a pharmaceutical composition comprising a therapeutically effective amount of a hydrohalide salt of Compound A in combination with a therapeutically effective amount of an agent useful for treating HIV infection and/or AIDS (alternatively referred to as an HIV/AIDS treatment agent) selected from:

(1) an HIV/AIDS antiviral agent,

(2) an anti-infective agent, and

(3) an immunomodulator.

The present invention also includes the use of a hydrohalide salt of Compound A as described above as a medicament for (a) inhibiting HIV protease, (b) preventing or treating infection by HIV, (c) delaying the onset of AIDS, or (d) treating AIDS. The present invention further includes the use of a hydrohalide salt of Compound A as described above in the preparation of a medicament for (a) inhibiting HIV protease, (b) preventing or treating infection by HIV, (c) delaying the onset of AIDS, or (d) treating AIDS.

The present invention also includes the use of a hydrohalide salt of Compound A of the present invention as described above in combination with one or more HIV/AIDS treatment agents selected from an HIV/AIDS antiviral agent, an anti-infective agent, and an immunomodulator for use as a medicament for (a) inhibiting HIV protease, (b) preventing or treating infection by HIV, (c) delaying the onset of AIDS, or (d) treating AIDS, said medicament comprising an effective amount of the hydrohalide salt of Compound A and an effective amount of the one or more treatment agents.

The present invention further includes the use of a hydrohalide salt of Compound A of the present invention as described above in combination with one or more HIV/AIDS treatment agents selected from an HIV/AIDS antiviral agent, an anti-infective agent, and an immunomodulator for the manufacture of a medicament for (a) inhibiting HIV protease, (b) preventing or treating infection by HIV, (c) delaying the onset of AIDS, or (d) treating AIDS, said medicament comprising an effective amount of the hydrohalide salt of Compound A and an effective amount of the one or more treatment agents.

For the uses described above, the hydrohalide salts of Compound A of the present invention may be administered orally, parenterally (including subcutaneous injections, intravenous, intramuscular, intrastemal injection or infusion techniques), by inhalation spray, or rectally, in dosage unit formulations containing conventional non-toxic pharmaceutically-acceptable carriers, adjuvants and vehicles.

The term “administration” and variants thereof (e.g., “administering” a compound) in reference to a hydrohalide salt of Compound A mean providing the salt to the individual in need of treatment. When a salt of the invention is provided in combination with one or more other active agents (e.g., AIDS antivirals), “administration” and its variants are each understood to include concurrent and sequential provision of the salt and other agents.

As used herein, the term “composition” is intended to encompass a product comprising the specified ingredients in the specified amounts, as well as any product which results, directly or indirectly, from combination of the specified ingredients in the specified amounts.

The expression “pharmaceutically acceptable” means that the carrier, diluent or excipient must be compatible with the other ingredients of the formulation and not deleterious to the recipient thereof.

The term “subject,” (alternatively referred to herein as “patient”) as used herein refers to an animal, preferably a mammal, most preferably a human, who has been the object of treatment, observation or experiment.

The term “therapeutically effective amount” as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated.

The pharmaceutical compositions of the present invention may be in the form of orally-administrable suspensions, capsules or tablets, nasal sprays, sterile injectible preparations, for example, as sterile injectible aqueous or oleagenous suspensions or suppositories.

When administered orally as a suspension, these compositions are prepared according to techniques well-known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The injectible solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the drug with a suitable nonirritating excipient, such as cocoa butter, synthetic glyceride esters of polyethylene glycols, which are solid at ordinary temperatures, but liquefy and/or dissolve in the rectal cavity to release the drug.

The Compound A salts of this invention can be administered orally to humans on an active ingredient basis in a dosage range of 0.01 to 1000 mg/kg body weight per day in a single dose or in divided doses. One preferred dosage range is 0.1 to 200 mg/kg body weight per day orally in a single dose or in divided doses. Another preferred dosage range is 0.5 to 100 mg/kg body weight per day orally in single or divided doses. For oral administration, the compositions are preferably provided in the form of tablets containing 1 to 1000 milligrams of the active ingredient, particularly 1, 5, 10, 15. 20, 25, 50, 75, 100, 150, 200, 250, 300, 400, 500, 600, 750, 800, 900, and 1000 milligrams of the active ingredient for the symptomatic adjustment of the dosage to the patient to be treated. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The present invention is also directed to combinations of the hydrohalide salts of Compound A of the present invention with one or more agents useful in the treatment of HIV infection and/or AIDS. For example, the Compound A salts of this invention may be effectively administered, whether at periods of pre-exposure and/or post-exposure, in combination with effective amounts of the HIV/AIDS antivirals, imunomodulators, antiinfectives, or vaccines, such as those in Table 1 as follows:

TABLE 1
HIV/AIDS ANTIVIRALS, IMUNOMODULATORS,
ANTIINFECTIVES, AND OTHER TREATMENTS

| | | |
|---|---|---|
| Drug Name | Manufacturer | Indication |

ANTIVIRALS

| | | |
|---|---|---|
| Amprenavir | Glaxo Wellcome | HIV infection, AIDS, |
| 141 W94 | | ARC |
| GW 141 | | (protease inhibitor) |
| Abacavir | Glaxo Welcome | HIV infection, AIDS, |
| GW 1592 | | ARC |
| 1592U89 | | (reverse transcriptase |
| | | inhibitor) |
| Acemannan | Carrington Labs | ARC |
| | (Irving, TX) | |
| Acyclovir | Burroughs Wellcome | HIV infection, AIDS, ARC, |
| | | in combination with AZT |
| AD-439 | Tanox Biosystems | HIV infection, AIDS, ARC |
| AD-519 | Tanox Biosystems | HIV infection, AIDS, ARC |
| Adefovir dipivoxil | Gilead Sciences | HIV infection |
| AL-721 | Ethigen | ARC, PGL, HIV positive, |
| | (Los Angeles, CA) | AIDS |
| Alpha Interferon | Glaxo Wellcome | Kaposi's sarcoma, HIV, in |
| | | combination w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| neutralizes pH | Concepts | |
| labile alpha aberrant | (Rockville, MD) | |
| Interferon | | |
| AR177 | Aronex Pharm | HIV infection, AIDS, ARC |
| beta-fluoro-ddA | Nat'l Cancer Institute | AIDS-associated diseases |
| BMS-232623 | Bristol-Myers Squibb | HIV infection, AIDS, |
| (CGP-73547) | Novartis | ARC |
| | | (protease inhibitor) |
| BMS-234475 | Bristol-Myers Squibb/ | HIV infection, AIDS, |
| (CGP-61755) | Novartis | ARC |
| | | (protease inhibitor) |
| CI-1012 | Warner-Lambert | HIV-1 infection |
| Cidofovir | Gilead Science | CMV retinitis, herpes, |
| | | papillomavirus |
| Curdlan sulfate | AJI Pharma USA | HIV infection |
| Cytomegalovirus | MedImmune | CMV retinitis |
| immune globin | | |
| Cytovene | Syntex | sight threatening CMV |
| Ganciclovir | | peripheral CMV |
| | | retinitis |
| Delaviridine | Pharmacia-Upjohn | HIV infection, AIDS, |
| | | ARC |
| | | (protease inhibitor) |
| Dextran Sulfate | Ueno Fine Chem. | AIDS, ARC, HIV |
| | Ind. Ltd. (Osaka, | positive asymptomatic |
| | Japan) | |
| ddC | Hoffman-La Roche | HIV infection, AIDS, ARC |
| Dideoxycytidine | | |
| ddI | Bristol-Myers Squibb | HIV infection, AIDS, ARC; |
| Dideoxyinosine | | combination with AZT/d4T |
| DMP-450 | AVID | HIV infection, AIDS, |
| | (Camden, NJ) | ARC |
| | | (protease inhibitor) |
| EL10 | Elan Corp, PLC | HIV infection |
| | (Gainesville, GA) | |
| Efavirenz | DuPont | HIV infection, AIDS, |
| | (SUSTIVA ®), | |
| (DMP 266) (−) | Merck | ARC |
| 6-Chloro-4(S)- | (STOCRIN ®) | (non-nucleoside RT |
| cyclopropylethynyl- | | inhibitor) |
| 4(S)-trifluoro- | | |
| methyl- | | |
| 1,4-dihydro-2H-3,1- | | |
| benzoxazin-2-one, | | |
| Famciclovir | Smith Kline | herpes zoster, herpes |
| | | simplex |
| FTC | Emory University | HIV infection, AIDS, ARC |
| | | (reverse transcriptase |
| | | inhibitor) |
| GS 840 | Gilead | HIV infection, AIDS, ARC |
| | | (reverse transcriptase |
| | | inhibitor) |
| HBY097 | Hoechst Marion | HIV infection, AIDS, ARC |
| | Roussel | (non-nucleoside reverse |
| | | transcriptase inhibitor) |
| Hypericin | VIMRx Pharm. | HIV infection, AIDS, ARC |
| Recombinant | Triton Biosciences | AIDS, Kaposi's sarcoma, |
| Human | | |
| Interferon Beta | (Almeda, CA) | ARC |
| Interferon alfa-n3 | Interferon Sciences | ARC, AIDS |
| Indinavir | Merck | HIV infection, AIDS, ARC, |
| | | asymptomatic HIV positive, |
| | | also in combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, AIDS, |
| | | ARC (reverse |
| | | transcriptase inhibitor); |
| | | also with AZT |
| Lobucavir | Bristol-Myers Squibb | CMV infection |
| Nelfinavir | Agouron | HIV infection, AIDS, |
| | Pharmaceuticals | ARC (protease inhibitor) |
| Nevirapine | Boeheringer | HIV infection, AIDS, |
| | Ingleheim | ARC (non-nucleoside |
| | | reverse transcriptase |
| | | inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, |
| | | HIV infection, |
| Phosphonoformate | Products, Inc | other CMV |
| | | infections |
| PNU-140690 | Pharmacia Upjohn | HIV infection, AIDS, ARC |
| | | (protease inhibitor) |
| Probucol | Vyrex | HIV infection, AIDS |
| RBC-CD4 | Sheffield Med. Tech | HIV infection, AIDS, |
| | (Houston TX) | ARC |
| Ritonavir | Abbott | HIV infection, AIDS, |
| (ABT-538) | | ARC (protease inhibitor) |
| Saquinavir | Hoffmann-LaRoche | HIV infection, AIDS, |
| | | ARC (protease inhibitor) |
| Stavudine; d4T | Bristol-Myers Squibb | HIV infection, AIDS, ARC |
| Didehydrodeoxy- | | |
| thymidine | | |
| Valaciclovir | Glaxo Wellcome | genital HSV & CMV |
| | | infections |
| Virazole | Viratek/lCN | asymptomatic HIV |
| Ribavirin | (Costa Mesa, CA) | positive, LAS, ARC |
| VX-478 | Vertex | HIV infection, AIDS, ARC |
| Zalcitabine | Hoffmann-La Roche | HIV infection, AIDS, ARC, |
| | | with AZT |
| Zidovudine; AZT | Glaxo Wellcome | HIV infection, AIDS, ARC, |
| | | Kaposi's sarcoma in |
| | | combination with other |
| | | therapies (reverse |
| | | transcriptase inhibitor) |
| ABT-378; Lopinavir | Abbott | HIV infection, AIDS, ARC |
| | | (protease inhibitor) |
| ABT-378/r; contains | Abbott | HIV infection, |
| | | AIDS, ARC |
| lopinavir and | | (protease inhibitor) |
| ritonavir; | | |
| Kaletra | | |
| JE2147/AG1776 | Agouron | HIV infection, AIDS, ARC |
| | | (protease inhibitor) |
| T-20 | Trimeris | HIV infection, AIDS, ARC |
| | | (fusion inhibitor) |
| T-1249 | Trimeris | HIV infection, AIDS, ARC |
| | | (fusion inhibitor) |
| BMS 232632 | Bristol-Myers-Squibb | HIV infection, AIDS, ARC |
| | | (protease inhibitor) |
| PRO 542 | Progenics | HIV infection, AIDS, ARC |
| | | (attachment inhibitor) |
| PRO 140 | Progenics | HIV infection, AIDS, ARC |
| | | (CCR5 co-receptor |
| | | inhibitor) |
| TAK-779 | Takeda | HIV infection, |
| | | AIDS, ARC |
| | | (injectable CCR5 receptor |
| | | antagonist) |
| DPC 681 & | DuPont | HIV infection, AIDS, ARC |
| DPC 684 | | (protease inhibitors) |
| DPC 961 & | DuPont | HIV infection AIDS, ARC |
| DPC 083 | | (nonnucleoside reverse |
| | | transcriptase inhibitors) |
| Trizivir (contains | GlaxoSmithKline | HIV infection, AIDS, ARC |
| abacavir, | | (reverse transcriptase |
| lamivudine, and | | inhibitors) |
| zidovudine) | | |
| tipranavir | Boehringer Ingelheim | HIV infection, AIDS, ARC |
| (PNU-140690) | (purchased from | (protease inhibitor) |
| | Pharmacia & Upjohn) | |
| tenofovir disoproxil | Gilead | HIV infection, AIDS, ARC |
| fumarate | | (reverse transcriptase |
| | | inhibitor) |
| TMC-120 & | Tibotec | HIV infections, |
| TMC-125 | | AIDS, ARC |
| | | (non-nucleoside reverse |
| | | transcriptase inhibitors) |
| TMC-126 | Tibotec | HIV infection, AIDS, ARC |
| | | (protease inhibitor) |

IMMUNO-MODULATORS

| | | |
|---|---|---|
| AS-101 | Wyeth-Ayerst | AIDS |
| Bropirimine | Pharmacia Upjohn | advanced AIDS |
| Acemannan | Carrington Labs, Inc. | AIDS, ARC |
| | (Irving, TX) | |
| CL246,738 | American Cyanamid | AIDS, Kaposi's sarcoma |
| | Lederle Labs | |
| EL10 | Elan Corp, PLC | HIV infection |
| | (Gainesville, GA) | |
| FP-21399 | Fuki ImmunoPharm | blocks HIV fusion with |
| | | CD4+ cells |
| Gamma Interferon | Genentech | ARC, in combination |
| | | w/TNF (tumor |
| | | necrosis factor) |
| Granulocyte | Genetics Institute | AIDS |
| Macrophage Colony | Sandoz | |
| Stimulating | | |
| Factor | | |
| Granulocyte | Hoeschst-Roussel | AIDS |
| Macrophage Colony | Immunex | |
| Stimulating | | |
| Factor | | |
| Granulocyte | Schering-Plough | AIDS, combination w/AZT |
| Macrophage Colony | | |
| Stimulating Factor | | |
| HIV Core Particle | Rorer | seropositive HIV |
| Immunostimulant | | |
| IL-2 | Cetus | AIDS, in combination |
| Interleukin-2 | | w/AZT |
| IL-2 | Hoffman-La Roche | AIDS, ARC, HIV, in |
| Interleukin-2 | Immunex | combination w/AZT |
| IL-2 | Chiron | AIDS, increase in CD4 cell |
| Interleukin-2 | | counts |
| (aldeslukin) | | |
| Immune Globulin | Cutter Biological | pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's sarcoma, |
| | (New Orleans, LA) | ARC, PGL |
| Imuthiol Diethyl | Merieux Institute | AIDS, ARC |
| Dithio Carbamate | | |
| Alpha-2 | Schering Plough | Kaposi's sarcoma w/AZT, |
| Interferon | | AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response | immunotherapeutic |
| | Corp. | |
| rCD4 | Genentech | AIDS, ARC |
| Recombinant | | |
| Soluble Human | | |
| CD4 | | |
| rCD4-IgG | | AIDS, ARC |
| hybrids | | |
| Recombinant | Biogen | AIDS, ARC |
| Soluble Human | | |
| CD4 | | |
| Interferon | Hoffman-La Roche | Kaposi's sarcoma, AIDS, |
| Alfa 2a | | ARC, in combination |
| | | w/AZT |
| SK&F106528 | Smith Kline | HIV infection |
| Soluble T4 | | |
| Thymopentin | Immunobiology | HIV infection |
| | Research Institute | |
| Tumor Necrosis | Genentech | ARC, in combination |
| Factor; TNF | | w/gamma Interferon |
| etanercept | Immunex Corp | rheumatoid arthritis |
| | (Enbrel ®) | |
| infliximab | Centocor | rheumatoid arthritis and |
| | (Remicade ®) | Crohn's disease |

ANTI-INFECTIVES

| | | |
|---|---|---|
| Clindamycin with | Pharmacia Upjohn | PCP |
| Primaquine | | |
| Fluconazole | Pfizer | cryptococcal meningitis, |
| | | candidiasis |
| Pastille | Squibb Corp. | prevention of |
| Nystatin Pastille | | oral candidiasis |
| Ornidyl | Merrell Dow | PCP |
| Eflornithine | | |
| Pentamidine | LyphoMed | PCP treatment |
| Isethionate | (Rosemont, IL) | |
| (IM & IV) | | |
| Trimethoprim | | antibacterial |
| Trimethoprim/sulfa | | antibacterial |
| Piritrexim | Burroughs Wellcome | PCP treatment |
| Pentamidine | Fisons Corporation | PCP prophylaxis |
| isethionate for | | |
| inhalation | | |
| Spiramycin | Rhone-Poulenc | cryptosporidia diarrhea |
| Intraconazole- | Janssen Pharm. | histoplasmosis; |
| R51211 | | cryptococcal |
| | | meningitis |
| Trimetrexate | Warner-Lambert | PCP |

OTHER

| | | |
|---|---|---|
| Daunorubicin | NeXstar, Sequus | Karposi's sarcoma |
| Recombinant | Ortho Pharm. Corp. | severe anemia assoc. with |
| Human | | AZT therapy |
| Erythropoietin | | |
| Recombinant | Serono | AIDS-related wasting, |
| Human | | cachexia |
| Growth Hormone | | |
| Leukotriene B4 | — | HIV infection |
| Receptor | | |
| Antagonist | | |
| Megestrol Acetate | Bristol-Myers Squibb | treatment of anorexia assoc. |
| | | w/AIDS |
| Soluble CD4 | — | HIV infection |
| Protein and | | |
| Derivatives | | |
| Testosterone | Alza, Smith Kline | AIDS-related wasting |
| Total Enteral | Norwich Eaton | diarrhea and malabsorption, |
| Nutrition | Pharmaceuticals | related to AIDS |

It will be understood that the scope of combinations of the Compound A salts of this invention with HIV/AIDS antivirals, immunomodulators, anti-infectives or vaccines is not limited to the list in Table 1 above, but includes in principle any combination with any pharmaceutical composition useful for the treatment of HIV infection and/or AIDS. When employed in combination with the compounds of the invention, the HIV/AIDS antivirals and other agents are typically employed in their conventional dosage ranges and regimens as reported in the art, including the dosages described in the Physicians' Desk Reference, 54^{th} edition, Medical Economics Company, 2000. The dosage ranges for a compound of the invention in these combinations are the same as those set forth above just before Table 1.

One suitable combination is a hydrohalide salt of Compound A of the present invention and a nucleoside inhibitor of HIV reverse transcriptase such as AZT, 3TC, ddC, or ddI. Another suitable combination is a Compound A salt of the present invention and a non-nucleoside inhibitor of HIV reverse transcriptase, such as efavirenz, and optionally a nucleoside inhibitor of HIV reverse transcriptase, such as AZT, 3TC, ddC or ddI.

Still another suitable combination is any one of the combinations in the preceding paragraph, further comprising an additional HIV protease inhibitor such as indinavir, nelfinavir, ritonavir, saquinavir, amprenavir, or abacavir. An aspect of this combination is the combination wherein the additional inhibitor of HIV protease is the sulfate salt of indinavir. Another aspect of this combination is the combination in which the additional protease inhibitor is selected from nelfinavir and ritonavir. Still another aspect of this combination is the combination in which the additional inhibitor of HIV protease is saquinavir, which is typically administered in a dosage of 600 or 1200 mg tid.

Other suitable combinations include a compound of the present invention with the following (1) efavirenz, optionally with AZT and/or 3TC and/or ddli and/or ddC, and optionally with indinavir; (2) any of AZT and/or ddl and/or ddC and/or 3TC, and optionally with indinavir; (3) d4T and 3TC and/or AZT; (4) AZT and 3TC; and (5) AZT and d4T.

Another aspect of the present invention is co-administration of a hydrohalide salt of Compound A of the present invention with an inhibitor of cytochrome P450 monooxygenase in an amount effective to improve the pharmacokinetics of the compound. Compound A can be metabolized, at least in part, by cytochrome P450 (CYP3A4). Co-administration of Compound A hydrohalide salts of the invention with a cytcochrome P450 inhibitor can improve the pharmacokinetic profile of Compound A in subjects (e.g., humans); i.e., co-administration can increase Cₘₐₓ (the maximum plasma concentration of the compound), AUC (area under the curve of plasma concentration of the compound versus time), and/or the half-life of Compound A. A suitable P450 inhibitor is ritonavir. It is to be understood that the primary role of ritonavir in this circumstance is as a pharmacokinetic modulator and not as a protease inhibitor; i.e., an amount of ritonavir which is effective for improving the pharmacokinetics of Compound A can provide a secondary or even negligible contribution to the antiviral effect.

A Compound A salt of the present invention can also be administered in combination with an HIV integrase inhibitor such as a compound described in WO 99/62520, WO 99/62513, or WO 99162897. A Compound A salt of the present invention can also be administered in combination with a CCR5 receptor antagonist, such as a compound described in WO 00/59502 or WO 00/59503.

In the above-described combinations, the Compound A hydrohalide salts of the present invention and other active agents may be administered together or separately. In addition, the administration of one agent may be prior to, concurrent with, or subsequent to the administration of other agent(s). These combinations may have unexpected or synergistic effects on limiting the spread and degree of infection of HIV.

Abbreviations used herein include the following:

ACN=acetonitrile

AIDS=acquired immune deficiency syndrome

Alloc or alloc=allyloxycarbonyl

ARC=AIDS related complex

BOC or Boc=t-butyloxycarbonyl

D=dimethylformamide

DSC=differential scanning calorimetry

EDC=1-ethyl-3-(3-dimethylaminopropyl)carbodiimide

Et=ethyl

HIV=human immunodeficiency virus

HOBT or HOBt=1-hydroxy benzotriazole hydrate

HPLC=high performance liquid chromatography

IPAc=isopropyl acetate

IPA=isopropyl alcohol

KF=Karl Fisher titration for water

LC=liquid chromatography

LHMDS=lithium hexamethyldisilazide

Me=methyl

MeOH=methanol

MSA=methanesulfonic acid

MTBE=methyl tert-butyl ether

NCS=N-chlorosuccinimide

NMR=nuclear magnetic resonance

NSA=naphthalenesuflonic acid

PPA=polyphosphoric acid

i-Pr=isopropyl

TBDC=di t-butyl dicarbonate

TEA=triethylamine

TGA=thermogravimetric analysis

THF=tetrahydrofuran

XRPD=x-ray powder diffraction

The following examples serve only to illustrate the invention and its practice. The examples are not to be construed as limitations on the scope or spirit of the invention.

3-Methoxy-5-bromopyridine (2)

| | |
|---|---|
| 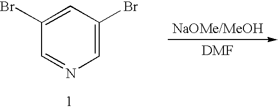 | 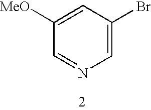 |

| Material | MW (g/mol) | Amount | | mmol |
|---|---|---|---|---|
| Dibromopyridine 1, 98% | 236.89 | 48.34 | g | 200 |
| Bromomethoxypyridine 2 | 188.02 | | | |
| 25 wt % NaOMe/MeOH | 54.02 | 56 | mL | 240 |
| (d = 0.945) | | | | |
| DMF | | 48 | mL | |
| 20% Sodium chloride | | 420 | mL | |
| Methyl-t-butyl ether | | 300 | mL | |
| Water | | 120 | mL | |

To a 500 mL flask equipped with condenser for distillation was charged 48.34 g dibromopyridine (200 mmol), 56 mL of 25 wt % NaOMe/CH₃OH (240 mmol) and 48 mL DMF. The mixture was heated to 90° C. A clear solution formed, which then turned cloudy. The mixture was aged 4 h at 90° C. and 3 h at 100° C. HPLC assay showed that the reaction was complete (<0.1A % of 1). Some solvent was distilled out continuously during the age to maintain the internal temperature. The mixture was cooled to 18° C. and 60 mL water and 60 mL 20% NaCl were added. After 1 min, 300 mL MTBE was added. The mixture was agitated for 5 min and was transferred to a separatory funnel. After settling, the bottom aqueous layer was cut and the organic was washed with 3×120 mL 20% NaCl, then 60 mL water. The assay yield of bromomethoxypyridine 2 was 88% as determined by HPLC.

¹H NMR (CDCl₃, 500 Hz): δ8.30 (s, 1 H), 8.25 (d, J=2.3 Hz, 1 H), 7.37-7.38 (m, 1 H), 3.87 (s, 3 H).

| | |
|---|---|
| HPLC Assay: | |
| Column: | Zorbax RX-C8 (4.6 mm × 250 mm) |
| Solvents: | 60% CH₃CN, 40% 0.1% H₃PO₄ |
| Flow: | 1.0 mL/min |
| Sample volume: | 10 μL |
| Wavelength: | 210 nm |
| Retention times: | |
| The tarry impurity | 2.1 min |
| Dimethoxypyridine | 2.4 min |
| DMF | 2.7 min |
| Bromomethoxypyridine 2 | 4.9 min |
| Dibromopyridine 1 | 6.2 min |

3-Methoxy-5-(t-butyloxycarbonylaminomethylcarbonyl)-pyridine (5)

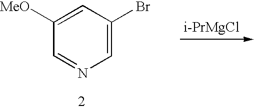

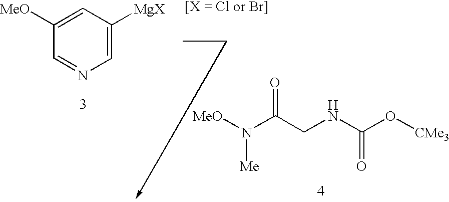

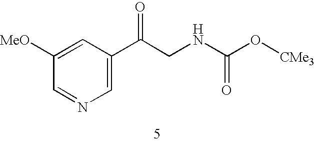

| Material | MW (g/mol) | Amount | | moles |
|---|---|---|---|---|
| Bromomethoxypyridine 2 | 188.02 | | | 18.9 |
| BOC-Aminoketone 5 | 266.29 | | | |
| iso-Propylmagnesium chloride/THF | | 17.6 | L | 35.2 |
| Weinreb amide 4, 96.3 wt % | 218.26 | 3.44 | Kg | 15.19 |
| Tetrahydrofuran | | 114 | L | |
| Isopropyl acetate | | 92 | L | |
| n-Heptane | | 45 | L | |
| 37% Hydrochloric acid | | 3.48 | Kg | 35.2 |
| Water | | 82 | L | |

A solution of 2 in MTBE was concentrated in a 100 L flask to 23 L by vacuum distillation at <40° C. About 91 L of dry THF was added slowly during distillation to solvent switch to THF and to dry the solution. The final THF solution (23 L, 18.9 mol 2) was charged into a 50 L flask, and the flask was degassed and then placed under a nitrogen atmosphere. After cooling over an ice bath, 10.4 L 2M iso-PrMgCl/THF was added over 21 min at <24° C. to afford a cloudy mixture. The mixture was aged 2 h at 20-30° C. at which point HPLC assay showed 2 at 1.0 A %. The reaction mixture after 1 h age was a clear, dark brown solution. The Grignard solution 3 was held 3 h more before its use.

In another 100 L flask, 3.44 kg Weinreb amide (15.19 mol) was mixed with 23 L dry THF. The cloudy solution was degassed and placed under an inert nitrogen atmosphere. The solution was cooled to −21° C. to give a slurry to which was added 7.2 L 2M i-PrMgCl/THF over 30 min at <−10° C. A nearly clear gray solution formed, to which the Grignard solution 3 was added over 30 minutes at <−11° C. The gummy precipitate that formed in the Grignard solution was not soluble in THF and was not transferred. The mixture was warmed to 24° C. over 1 h and the resulting dark red solution was aged 15 h at 15-24° C. HPLC assay showed that the reaction complete (<1A % Weinreb amide 4). The dark reddish-brown mixture was cooled to 9° C., and 23 L dilute aqueous HCl (prepared using 3.48 kg of 37 wt % HCl) was added with vigorous agitation at <35° C. The mixture was agitated for 5 min, transferred to an 100 L extractor, allowed to settle, and the bottom aqueous layer (pH 7-8) was cut. The organic layer was transferred back to the 100 L flask where it was batch concentrated to 55 L at <40° C. and flushed with 90 L of IPAc to solvent switch. The final solution concentrate (˜55 L) was cooled to room temperature and diluted with 23 L water. The mixture was transferred to a 100 L extractor, allowed to settle, and the aqueous layer was cut. The organic layer was washed with 2×23 L and 1×16 water. The organic layer was batch concentrated at <35° C. in a 72 L flask. The final concentration to 13 L was done at <60° C. The mixture was then heated to 64° C. to form a clear solution, then cooled to 58° C. at which point 3 g of seed crystals of 5 were added. A slurry formed at 55° C. The slurry was cooled to 25° C., 39 L of n-heptane was added over 40 min, and the slurry was aged 15 h at room temperature and then 2 h at 0-5° C. The solids were filtered, rinsed with 8 L 3:1 n-heptane/IPAc and dried in a vacuum oven at 50° C. to afford 3.61 Kg of 5 as a yellowish crystalline solid (87% yield based on 4, 99.7 A % and 97.3 wt % purity).

¹H NMR (CDCl₃, 500 Hz): δ8.77 (s, 1 H), 8.53 (d, J=2.8 Hz, 1 H), 7.70-7.71 (m, 1 H), 5.49 (broad s, 1 H), 4.48 (d, J=4.1 Hz, 2 H), 3.93 (s, 3 H), 1.49 (s, 9 H).

| | |
|---|---|
| HPLC Assay: | |
| Column: | Zorbax RX-C8 (4.6 mm × 250 mm) |
| Solvents: | 60% CH₃CN, 40% 0.1% H₃PO₄ |
| Flow: | 1.0 mL/min |
| Sample volume: | 10 μL |
| Wavelength: | 210 nm |
| Retention times: | |
| Methoxypyridine | 2.2 min |
| Dimethoxypyridine | 2.4 min |
| Weinreb amide 4 | 3.4 min |
| BOC-aminoketone 5 | 3.9 min |
| IPAc | 4.1 min |
| Bromomethoxypyridine 2 | 4.9 min |
| Impurity | 6.2 min |

3-Methoxy-5-(aminomethylcarbonyl)pyridine HCl salt

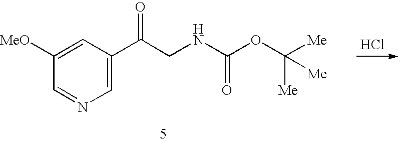

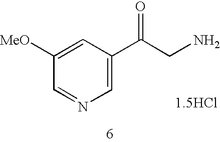

| Material | MW (g/mol) | Amount | | moles |
|---|---|---|---|---|
| BOC-Aminoketone 5 | 266.29 | 2.19 | Kg | 8.22 |
| 5N HCl | | 4.93 | L | 24.6 |
| 5N NaOH | | 2.47 | L | 12.3 |

To a 22 L round bottomed flask equipped with an overhead stirrer, thermocouple probe and nitrogen line was charged 4.93 L 5N HCl. The acid solution was warmed to 40° C. over a steam bath, after which 2.19 Kg of solid BOC-aminoketone 5 was added in portions over 20 min. After the addition, the reaction solution was aged 1.3 h at 40° C. Ice was then added to the bath to cool the batch to 15° C. and 2.47 L 5N NaOH was added over 50 min to neutralize the excess HCl. The resulting solution was cooled over salt/ice bath.

hu 1H NMR (D₂O, 500 Hz): δ8.96 (s, 1 H), 8.73 (d, J=2.5 Hz, 1 H), 8.52-8.53 (m, 1 H), 4.77 (s, 2 H), 4.07 (s, 3 H).

| HPLC Assay: | | |
|---|---|---|
| Column: | Zorbax RX-C8 (4.6 mm × 250 mm) | |
| Gradient: | min | CH₃CN/0.1% H₃PO₄ |
| | 0 | 5/95 |
| | 12 | 90/10 |
| | 13 | 5/95 |

| | |
|---|---|
| Flow: | 1.0 mL/min |
| Sample volume: | 5 μL |
| Wavelength: | 210 nm |
| Retention times: | |
| Aminoketone 6 | 2.1 min |
| BOC-aminoketone 5 | 8.2 min |

### EXAMPLE 4

### 4-(tert-butyloxycarbonyl)-2(S)-((2,2,2-trifluoroethyl)aminocarbonyl)piperazine

Step One: Preparation of the Pyrazine Amide

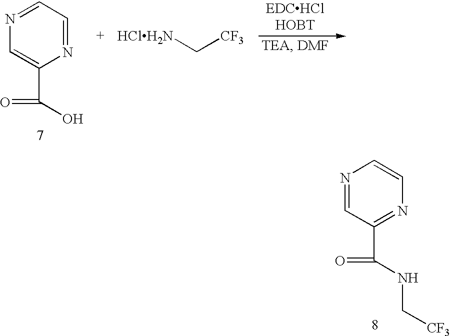

Pyrazine 2-carboxylic acid (1204 g) was suspended in DMF (4.8 L, 4 mL/g acid). 2,2,2-trifluoroethylamine.HCl (TFEA.HCl) (1200 g), 1-hydroxybenzotriazole (HOBT) (60 g) and triethylamine (TEA) (1410 mL) were then added sequentially (exotherm upon addition of TEA, flask cooled with ice bath and temperature kept below 35° C). The reaction was cooled to 15° C. and 1-(3--dimethylaminopropyl)-3-ethylcarbodiimide.HCl (EDC.HCl) (1940 g) was added portionwise over 15-30 min. The reaction temperature was kept below 35° C. When the reaction appeared complete (approx. two hours, <5% pyrazine 2-carboxylic acid by LC assay), the reaction mixture (yellow/white slurry) was diluted with 10% K₂CO₃ in water (24 L, 20 mL/g acid) and the reaction slurry was kept below 35° C. The slurry was cooled to 10° C., aged for two hours and filtered (mother liquor assay=3-4 mg/mL). The wet cake was washed with deionized water (12 L, 10 mL/g acid) and dried under vacuum (22″ Hg) at 40° C. with a nitrogen purge. Theoretical yield of 1816 g. Actual yield 1533 g (84%).

¹H NMR: (CD₃CN, 400 MHz): δ9.29(d, J=1.5 Hz, 1H), 8.82 (d, J=2.5 Hz, 1H), 8.63 (dd, J=2.6,1.4 Hz, 1H), 8.40 (bs, 1H), 4.14 (dq, J=9.4, 6.8 Hz, 2H).

HPLC Assay conditions: Waters Xterra RP8 column, elution with acetonitrile and 5 mM K phosphate adjusted to pH=8, detection at 220 nm.

Step Two: Preparation of the Piperazine Amide

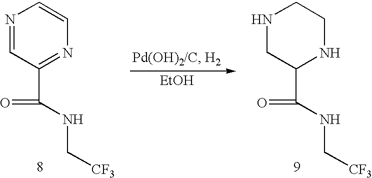

Pyrazine amide (60.2 g 0.268 mol, not corrected for water content) was suspended in absolute ethanol (550 mL) in a 1.0 L autoclave hydrogenation vessel and cooled to 15° C. Wet 20% Pd(OH)₂/C 11.0 g (20 wt %, 50 wt % wet) was added and reaction was purged with N₂ three times. H₂ (5 psig) was introduced with stirring and the temperature maintained at 15° C. for 60 minutes. The temperature was then increased to 60° C. and the hydrogen pressure increased to 40 psig and the reaction mixture stirred for 18 additional hours. The reaction was considered complete when conversion is >99% by LC assay. The reaction mixture was filtered through Solka-Floc and the catalyst solids were washed with ethanol 2×110 mL. Assay of the combined filtrate and washes gave 53.5 g of racemic piperazine amide (Yield=86%)

¹H NMR (CD3CN, 400 MHz): δ7.58 (bs, 1H), 3.90 (dq, J=9.5,6.7 Hz, 2H), 3.24(dd, J=7.9, 5.5 Hz, 1H), 2.96 (dd, J=12.1, 3.6 Hz, 1H), 2.84-2.78 (m, 1H), 2.77-2.67 (m, 3H), 2.66-2.56 (m, 1H), 1.90 (s, 2 H).

HPLC Assay conditions: YMC Basic column, elution with acetonitrile and 0.1% aqueous H₃PO₄, detection at 210 nm.

Step Three: Resolution of the Piperazine Amide

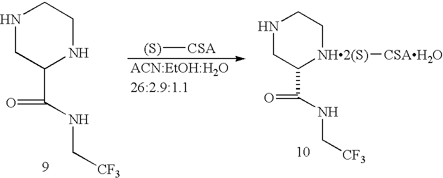

The pip amide ethanol filtrate (116.37 g containing 10.3 g of racemic pip amide by LC assay) was concentrated in vacuo to a final volume of 40.2 mL (3.9 mL per gram of pip amide) and the slurry is diluted with 82.4 mL (8 mL per gram pip amide) of acetonitrile (ACN) and stirred until homogenous. Separately (S)-camphorsulfonic acid ((S)-CSA) (19.26 g, MW=232.30, 1.7 eq) was dissolved in 185 mL of ACN (18 mL per gram of pip amide). The water content of the two solutions was then determined by Karl Fisher titration. The CSA solution was added to the pip amide solution giving a small exotherm to approx. 31-32° C. Water (11.02 mL, 1.118 mL per gram of pip amide minus the total water content of the two solutions) was then added, such that the acetonitrile:ethanol:water ratio was 26:2.9:1.1 (v/v/v). Solids began to form after 15-30 min. The solution/slurry was heated to 72° C. to completely dissolve all solids. The yellow solution was recooled to 62° C. and seeded with a slurry of 10.3 mg of pip amide salt in 1 mL of acetonitrile. After a two hour age at 62° C. the slurry was allowed to cool to room temperature overnight (crystallization was complete when loss to mother liquors was <21 mg pip amide/mL by LC assay. The slurry was filtered then washed with 2×30 mL of ACN:EtOH:H₂O [(26:2.9:1.1), (v:v:v)] solution. The wet cake (˜13 g, white solid) was dried at 40° C. in a vacuum oven (24 in Hg, nitrogen sweep) to give 11.16 g of product (yield=33%). Assay method (Pip Amide) as above. Chiral assay gives an enantiomeric excess (ee) of 98.0%.

1H NMR (CD₃OD, 400 MHz): d4.84(bs, 5H), 4.64 (dd, J=12.0, 3.6 Hz, 1H), 4.13-3.94 (m, 3H), 3.77 (m, 2H), 3.66 (m, 1H), 3.54-3.43 (m, 2H), 3.28(d, J=14.7 Hz, 2H), 2.82 (d, 14.7 Hz, 2H), 2.55 (m, 2H), 2.36 (m, 2H), 2.12-1.998 (m, 4H), 1.92 (d, J=18.4 Hz, 2H), 1.72 (m, 2H), 1.45 (m, 2H), 1.09 (s, 6H), 0.87 (s, 6H).

Enantiomeric excess determined by chiral HPLC of the mono BOC piperazine amide. BPLC assay conditions: Chiral AGP column, elution with acetonitrile and 10 mM Kphospate, pH=6.5, detection at 210 nm.

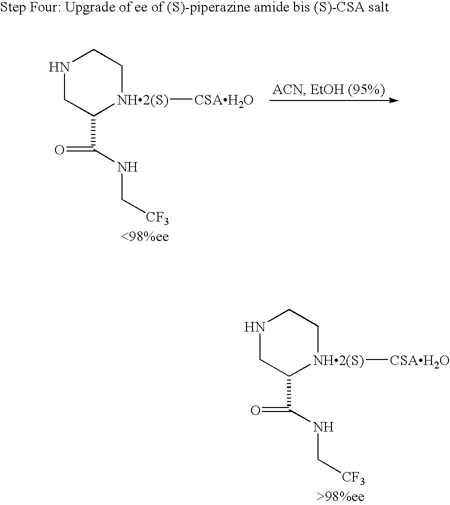

To a 12 L flask was charged (S)-pip amide salt (412.87 g) having an ee of less than 98%, 7.43 L of ACN and 825 mL of 190 proof EtOH. The slurry was heated to 75° C., aged for 1 hr at 75° C. (during heating the slurry thickened considerably), then allowed to cool to 25° C. overnight. The slurry was filtered and washed with EtOH (190 proof):ACN (10:90) (2×800 mL, 2 mL/g). The white solid was dried in a vacuum oven at 24 in Hg, 40° C. with a nitrogen sweep to give 400 g of product with an ee of 99%. Assays (normal and chiral) were performed as described above in the prior steps.

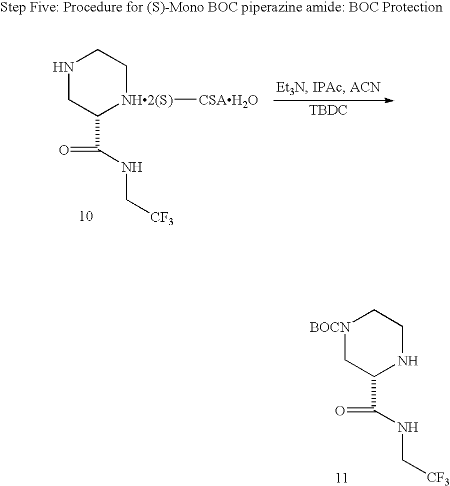

Bis (S)-CSA piperazine amide salt (20 g) was suspended in a mixture of 113 mL of isopropyl acetate (IPAc) and 57 mL of acetonitrile. Triethylamine (8.26 mL, 2 eq) was added and the mixture stirred until homogenous. A solution of di t-butyl dicarbonate (TBDC) (6.46 g, 1.0 eq) in a mixture of 20 mL isopropyl acetate and 10 mL of acetonitrile (ACN) was then added over 10 minutes. After aging for two hours the solution was assayed as necessary by LC (Pip Amide Assay, see above) until the reaction was complete (i.e., less than 5% starting material). When the reaction was complete, 100 mL of water and 135 mL of isopropyl acetate were added, the resulting layers were separated and the organic layer was concentrated to 28 mL. The residue was then diluted with 28 mL of isopropyl alcohol and reconcentrated to 28 mL. This was repeated two additional times. The yield of BOC pip amide was 87% with a mono:bis BOC ratio of 95:5, as determined by HPLC.

¹H NMR (CDCl₃, 400 MHz): δ=7.39 (app t, J=6.3 Hz, 1H), 3.96 (dd, J=3.5, 13.4 Hz, 1H), 3.88 (m, 2H), 3.67 (d, J=11.5 Hz, 1H), 3.39 (dd, J=3.8, 8.6 Hz, 1H), 3.13 (dd, J=8.6, 13.3 Hz, 1H), 3.02 (br, 1H), 2.91 (m, 1H), 2.77 (m, 1H), 1.43 (s, 9H).

¹³C NMR (CDCl₃, ) δ=171.43, 154.41, 123.89 (q, J=78.5 Hz), 80.16, 57.65, 43.63, 45.6 (br), 44.0 (br), 40.20 (q, J=34.7 Hz), 28.19.

HPLC Assay conditions: YMC Basic column, elution with acetonitrile and 0.1% aqueous H₃PO₄, detection at 210 nm.

Boc Alloc Piperazine 12

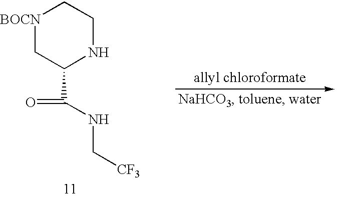

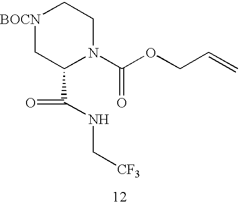

| Material | MW (g/mol) | Amount | | moles |
|---|---|---|---|---|
| Boc piperazine 11 | 311.3 | 15 | Kg | 16.1 |
| (34% soln. in IPA) | | | | |
| Toluene (flush) | | 38 | L | |
| Toluene (solvent) | | 33 | L | |
| Water | | 32.2 | L | |
| NaHCO₃ | 84 | 1.8 | Kg | 21.4 |
| Allyl Chloroformate | 120.5 | 2.2 | Kg | 18.5 |
| NaCl | | 360 | grams | |

To a 100 L batch concentrator equipped with condenser for distillation was charged 15 Kg of Boc Pipamide (34% soln.) and 38 L toluene. The solution was distilled under vacuum (40 C., 20 mm Hg) to solvent switch IPA for toluene. Expected distillate is 37 Kg or 44 L of mixed solvent. The vessel was then set for reaction and 33 L toluene (solvent), 25 L water, and 1.8 Kg NaHCO₃ were added. The batch was cooled to 15 C. and with vigorous mixing allyl chloroformate (18.5 L) was added by addition funnel at a rate to maintain batch temperature between 15-20° C. The reaction was mildly exothermic and reached about 20° C. by the end of the addition and remained at about that temperature for the duration of the reaction. When the reaction was complete as determined by HPLC, the agitation was stopped and the layers separated. The organic layer was washed with a solution of 360 grams NaCl in 7.2 L water.

¹H NMR (CDCl₃, 400 MHz) 5.95 (m, 1H), 5.35 (d, 1H), 5.28 (d, 1H), 4.75 (s, 1H), 4.68 (d, 1H), 4.53 (d, 1H), 3.90 (m, 3H), 3.20 (dd, 1H), 3.00 (m, 1H), 1.45 (s, 9H).

Alloc Piperazine 13

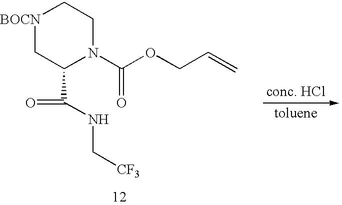

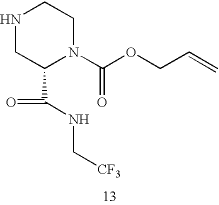

| Material | MW (g/mol) | Amount | | moles |
|---|---|---|---|---|
| Boc alloc piperazine 12 | 395.4 | approx. 34 kg | | 16.1 |
| | | solution | | |
| Conc. HCl | | 3.2 | L | 38.4 |
| Water | | 38 | L | |
| THF | | 54 | L | |
| Na₂CO₃ | 106 | 3.4 | Kg | 32.1 |
| NaCl | | 4.9 | Kg | |

Concentrated HCl (3.2 L) was added to a vigorously stirred solution of boc alloc piperazine 12 in toluene and the mixture was heated to 40° C. When the reaction was complete (in 1-2 hours as determined by HPLC), the reaction mixture was cooled to room temperature, and then water (33 L) was added. The batch was then cooled to 15° C. and THF (27 L) was added while maintaining the temperature at below 20° C. Na₂CO₃ (3.4 Kg) was then added in portions as it dissolved, followed by addition of NaCl (4.9 kg) to separate the organic from the aqueous layer. The organic layer was saved and the water layer was extracted a second time with THF (27 L).

¹H NMR (CDCl₃, 400 MHz) 7.28 (s, 1H), 4.00 (dd, 1H), 3.97 (m, 2H), 4.70 (s, 1H), 3.40 (dd, 1H), 3.20 (dd, 1H), 3.05 (s, 1H), 2.93 (d, 1H), 2.81 (t, 1H), 1.80 (s, 1H), 1.43 (s, 9H).

Piperazine Acid 14

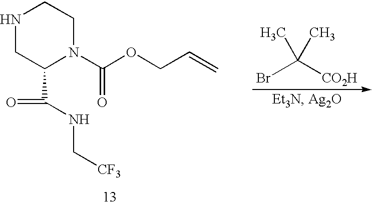

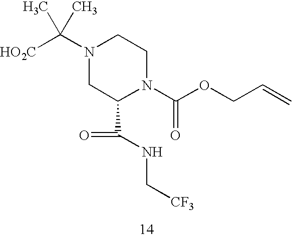

| Material | MW (g/mol) | Amount | | moles |
|---|---|---|---|---|
| Alloc piperazine 13 | 295.26 | 4.31 | Kg | 14.6 |
| 2-bromo-2-methyl- | 167.0 | 2.92 | Kg | 17.5 |
| propionic acid | | | | |
| triethylamine | 101.19 | 8.14 | L | 58.4 |
| Solka-floc | | 3 | Kg | |
| Silver oxide | 232 | 1.87 | Kg | 8.06 |
| Toluene | | 44 | L | |
| 6N HCl | | 38.4 | L | |
| 2N HC1 | | 39 | L | |

To a 100 L batch concentrator equipped with a condenser for distillation was charged alloc piperazine 13 (4.31 Kg, approximately a 7% solution in THF). The solution was distilled under vacuum to solvent switch THF for toluene while azeotroping residual water, after which a 100 L round bottomed flask was charged with the toluene solution and diluted with further toluene to a 10% solution. 2-bromo-2-methylpropionic acid (2.92 Kg) was then added, and the mixture was stirred until all material went into solution. Triethylamine (8.14 L) was then added followed by Solka-floc (2.97 Kg). The mixture was cooled while stirring with an ice water bath to 10° C., and then silver oxide was added in portions while maintaining the exothermic reaction at less than 40° C. After completion of the reaction (approximately one hour, as determined by EPLC), the reaction mixture was cooled with an ice bath and 6N HCl (38.4 L) was added in portions while maintaining the exothermic reaction at less than 50° C. The mixture was stirred for about ten minutes and filtered through a bed of Solka-floc. The filter cake was washed with 2N HCl (3×13 L). Further product was obtained from the combined filtrate and washings by cooling and separating the layers, adjusting the pH of the separated aqueous layer to 2 with aqueous NaOH, washing the layer with MTBE, separating the layers, adjusting the pH of the aqueous layer to 10 with aqueous NaOH, washing the layer with IPAc, separating the layers and adjusting the pH of the aqueous layer to 6 with 2N HCl, adding NaCl, and extracting the aqueous layer with THF.

¹H NMR (CDCl₃, 400 MHz): δ9.02 (broad s, 1 H), 6.98 (broad s, 1 H), 5.90-6.00 (m, 1 H), 5.24-5.28 (m, 2 H), 5.65 (broad s, 1 H), 4.16 (broad s, 1 H), 2.90-2.93 (m, 1 H), 2.55-2.57 (m, 1 H), 2.40-2.41 (m, 1 H), 1.59-1.61 (m,1H).

### EXAMPLE 8

### 4-[1-[5-(5-methoxy-3-pyridinyl)-carbonylmethylaminocarbonyl]-1-methylethyl]-2(S)-[(2,2,2-trifluoroethyl)aminocarbonyl]-1-[allyloxycarbonyl]piperazine, bis sulfate 15

4-[1-[5-(5-methoxy-3-pyridinyl)-carbonylmethylaminocarbonyl]-1-
methylethyl]-2(S)-[(2,2,2-trifluoroethyl)aminocarbonyl]-1-
[allyloxycarbonyl]piperazine, bis sulfate 15

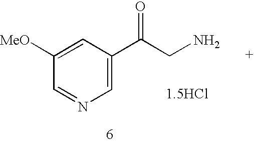

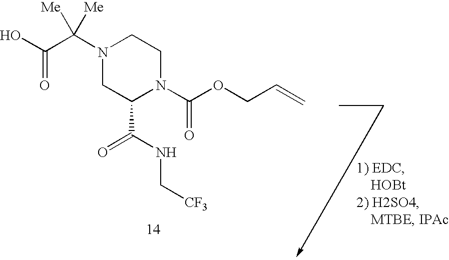

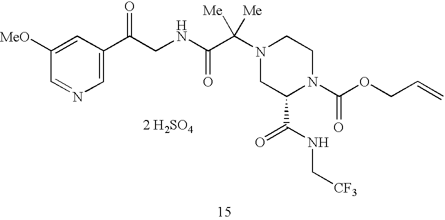

| Material | MW (g/mol) | Amount | | moles |
|---|---|---|---|---|
| Aminoketone solution 6 | | | | 8.22 |
| Piperazine Acid 14 | 381.35 | 3.13 | Kg | 8.22 |
| Tetrahydrofuran | | 15 | L | |
| Dimethylformamide | | 12.5 | L | |
| HOBt | 135.13 | 1.22 | Kg | 9.04 |
| EDC | 191.71 | 1.73 | Kg | 9.04 |
| Diisopropylethylamine | 129.25 | 4.29 | L | 24.6 |
| Ethyl Acetate | | 25 | L | |
| Sat'd NaHCO₃ | | 12.5 | L | |
| Water | | 13 | L | |
| Isopropyl Acetate | | 100 | L | |
| Sulfuric Acid (96 wt %, d = 1.84) | 98.08 | 0.786 | L | 14.2 |
| MTBE | | 42 | L | |

A 100 L round bottomed flask was fitted with a batch concentrator and 16.75 Kg solution (18.7 wt %, 8.22 mol) of piperazine acid 14 in THF was charged and batch concentrated. The solution was flushed with 2×7.5 L THF to dryness and was concentrated to a minimum volume. DMF (12.5 L) was added and residual THF was distilled at <25° C. HOBT hydrate (1.22 Kg) was added and allowed to dissolve. The batch was cooled to 17° C., and then 1.73 Kg EDC was added over 10 min. The resulting solution was aged 2 h at 21° C. The solution was cooled over a salt/ice bath to 0° C. and aminoketone hydrochloride 6 solution at 4° C. was added rapidly, followed by a 0.4 L water rinse. Following an exotherm to 16° C. and cooling over 20 min to 10° C., 4.29 L Hunig's base was added rapidly. With continued cooling over ice, the reaction mixture was aged 2.5 h. The mixture was diluted with 25 L EtOAc, 12.5 L sat'd NaHCO₃ and 6.3 L water and was cooled and aged 30 min to form a slurry. The precipitated HOBT was filtered on a filter pot and the cake was rinsed with a mixture of 8 L water and 1 L EtOAc. The filtrate was pumped into a 100 L extractor, cooled to 7° C. and acidified to pH 5 with 3.1 L 5N HCl. The aqueous layer was allowed to settle 30 min and was separated and extracted with 13 L EtOAc (0.4% product loss to aqueous layer). The combined organic layer was washed with 12 L sat'd NaHCO₃ (0.2% loss) and then with 12 L water (0.1% loss). The washed organic layer was weighed and assayed to contain 3.7 Kg ketoamide 15 (86% yield). This solution was batch concentrated to a minimal volume in a 100 L round bottomed flask, flushed with 3×10 L IPAc to dry and solvent switched, filtered through a glass funnel and adjusted to 19 L in IPAc. The solution was diluted with 19 L MTBE.

A 22 L round bottomed flask was charged with 19 L MTE and cooled over a salt/ice bath to 5° C. H₂SO₄ (0.786 L, 2 eq vs. ketoamide) was added over 20 min (exotherm to 13° C.). The 100 L round bottomed flask containing the ketoamide solution was fitted with a 5 L addition funnel, H₂SO₄/MTBE was pumped in portions to the funnel and added over 2 h. The slurry is filtered over a large filter pot, rinsed with 6 L 2:1 MTBE/IPAc and dried under nitrogen, then 2 days in a vacuum oven (40° C.) to afford 5.41 Kg bis-sulfate salt in 84% yield (67.8 wt % free base, 93.5 A %).

¹H NMR of free base (CDCl₃, 500 Hz): δ8.80 (s, 1 H), 8.52-8.53 (m, 1 H), 8.37 (broad s, 0.6 H), 7.90 (Broad s, 0.4 H), 7.70-7.71 (m, 1 H), 6.70 (broad s, 1 H), 5.94 (broad s, 1 H), 5.27-5.41 (m, 2 H), 4.87 (broad s, 1 H), 4.82 (s, 1 H), 4.67 (d, J=4.5 Hz, 2 H), 4.57 (d, J=18.3 Hz, 1 H), 4.05-4.14 (m, 2 H), 3.92 (s, 3 H), 3.66 (d, J=11.2 Hz, 1 H), 3.20-3.30 (m, 1 H), 2.90 (d, J=10.8 Hz, 1 H), 2.45 (d, J=9.3 Hz, 1 H), 2.35-2.37 (m, 1 H), 1.28 (s, 6 H).

| HPLC Assay: | | |
|---|---|---|
| Column: | Zorbax RX-C8 (4.6 mm × 250 mm) | |
| Gradient: | min | CH₃CN/0.1% H₃PO₄ |
| | 0 | 5/95 |
| | 12 | 90/10 |
| | 13 | 5/95 |

| | |
|---|---|
| Flow: | 1.0 mL/min |
| Sample volume: | 5 μL |
| Wavelength: | 210 nm |
| Retention times: | |
| HOBT | 4.8 min |
| Pyrazine dimer | 5.6 min |
| Piperazine acid 14 | 5.9 min |
| EtOAc | 6.3 min |
| Ketoamide 15 | 8.2 min |
| HOBT adduct | 11.1 min |

### EXAMPLE 9

### 4-[1-[5-(5-methoxy-3-pyridinyl)-2-oxazolyl]-1-methylethyl]-2(S)-[(2,2,2-trifluoroethyl)aminocarbonyl]-piperazine 16

4-[1-[5-(50methoxy-3-pyridinyl)-2-oxazolyl]-1-methylethyl]-2(S)-[(2,2,2-
trifluoroethyl)aminocarbonyl]-piperazine 16

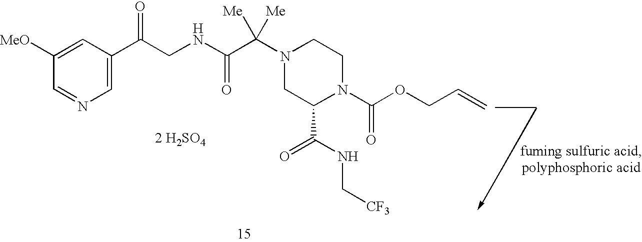

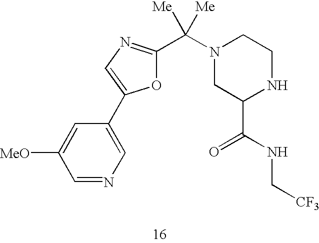

| Material | MW (g/mol) | Amount | | moles |
|---|---|---|---|---|
| Ketoamide 15 (67.8 wt % free base) | 529.51 | 3.57 | Kg | 6.74 |
| Polyphosphoric Acid | 342.04* | 7.67 | Kg | 22.4 |
| 30% Oleum (26-29%) | | 8.95 | Kg | |
| Water | | 32 | L | |
| 50% NaOH | | 30 | L | |
| IPAc | | 53 | L | |
| Brine | | 16 | L | |

*Estimated based on P₄O₁₀ equivalent.

To a 72 L round bottomed flask equipped with an overhead stirrer, thermocouple probe and nitrogen line was charged 8.95 L of 30% fuming sulfuric acid (oleum). The liquid was cooled over dry ice/acetone bath. When the temperature dropped to 9° C., crystallization of SO₃ was observed with concomitant exotherm to 11° C. Neat PPA (7.67 Kg viscous liquid) was then poured into the oleum over 1 h, avoiding the walls of glassware. A mild exotherm (−5° C.) was observed during addition. Ketoamide bis-sulfate salt 15 (5.26 Kg dusty powder) was added via funnel over 75 min at <20° C. to form a thick mixture with some undissolved ketoamide salt. The bath was drained and the mixture was heated over steam bath to 40-45° C. and aged 7 h to afford a homogeneous brown liquid. Reaction was complete as determined by HPLC assay. The reaction mixture was cooled over dry ice acetone bath to 3° C. and 22 L water was added slowly from an addition funnel to quench the reaction mixture, keeping the internal temperature <23° C. The solution was neutralized partially with 20 L 50% NaOH (<33° C.) and pumped into a 100 L Buchi reactor along with a 10 L water rinse, where it was adjusted to pH 1.7 with more 50% NaOH, diluted with 25 L IPAc, and then adjusted to pH 9.3 with 50% NaOH. The internal temperature was allowed to rise to 37° C. toward the end of addition to keep the salts solubilized. The two phases were separated and collected in polyjugs. Aqeuous loss was 0.2%. The Buchi vessel was thoroughly rinsed with water to remove gummy residues. The combined organic layer was washed with 16 L brine (0.04% loss) and 2 L water (0.2% loss). Assay yield was 2.44 Kg (85%) of title product 16 (90.4 A %, 95.1% ee).

¹H NMR (CDCl₃, 500 Hz): δ8.63 (broad s, 1 H), 8.50 (s, 1 H), 8.28 (s, 1 H), 7.36-7.37 (m, 1 H), 7.34 (s, 1 H), 3,91-4.00 (m, 2 H), 3.94 (s, 3 H), 3.78-3.79 (m, 1H), 3.02-3.11 (m, 4 H), 2.88-2.91 (m, 1 H), 2.65-2.73 (m, 2 H), 1.60 (s, 3 H), 1.59 (s, 3 H)

| HPLC Assay: | | |
|---|---|---|
| Column: | Zorbax RX-C8 (4.6 mm × 250 mm) | |
| Gradient: | min | CH₃CN/0.1% H₃PO₄ |
| | 0 | 5/95 |
| | 12 | 90/10 |
| | 13 | 5/95 |

| | |
|---|---|
| Flow: | 1.0 mL/min |
| Sample volume: | 5 μL |
| Wavelength: | 210 nm |
| Retention times: | |
| Ketoamide 15 | 5.3 min |
| Biarylpiperazine 16 | 5.6 min |

Tris-naphthalenesulfonic acid salt of 16
Part A - Purification of 2-Naphthalenesulfonic acid

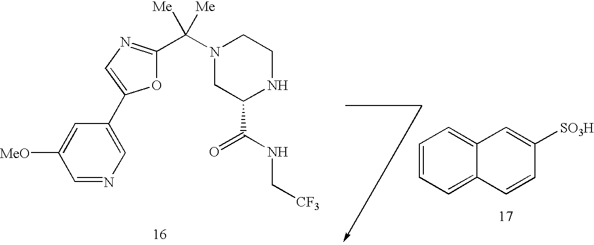

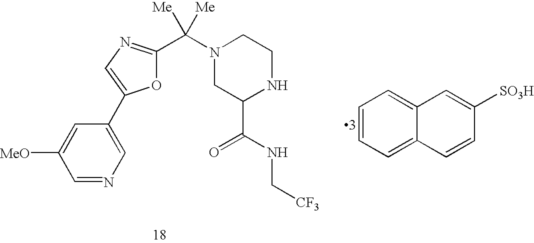

| Run 1 - | | | | |
|---|---|---|---|---|
| Material | MW (glmol) | Amount | | mmoles |
| 2-NSA 17 (76 wt % and 88A %) | 208.24 | 200 | g | 730 |
| 2-NSA 17 seed | | 0.30 | g | |
| Acetonitrile | | 800 | mL | |
| Water | | 10 | mL | |
| Toluene | | 950 | mL | |

The impurities present in the crude 2-NSA 17 included 1-NSA, naphthalene, two isomers of naphthalenesulfone, and sulfuric acid. The crude 2-NSA 17 (200 g) was mixed with 400 mL CH₃CN, 10 mL water and 800 mL toluene and heated to 78-80° C. to dissolve the solids. The two layers were allowed to settle and the lower black layer (about 100 mL) was cut at 80° C. The top layer was cooled and seeded at 40° C. (100 mg seed). A slurry formed at ˜33° C. The slurry was cooled to 6° C., rinsed with 150 1nL toluene and air dried in the funnel to afford 169 g of acid 17. In the black cut, most of 1-naphthalenesulfonic acid and sulfuric acid were rejected. In the mother liquor most of naphthalene and isomers of naphthalenesulfone are rejected. The purity of the filtered crystals was ˜98.6 A %.

The crystals were mixed with 340 mL CH₃CN and heated to 50° C. to form a clear, gray solution, which was cooled and seeded at 40° C. (200 mg seed). A slurry formed at ˜26° C. This was cooled to 5° C., filtered and rinsed with 100 mL CH₃CN to afford after drying in a vacuum oven at 60° C., 76.8 g solid (99.8 A %, 94.3 wt.% with 8% water, 48% recovery based on 76 wt % pure crude acid).

¹H NMR of 17 (DMSO-d6, δ) 8.17 (s, 1H), 7.98˜7.96 (m, 1H), 7.91˜7.90 (m, 1H), 7.88˜7.86 (m, 1H), 7.73˜7.71 (m, 1H), 7.53˜7.51 (m, 2H), 6.98 (broad, 3H).

| | |
|---|---|
| HPLC Assay: | |
| Column: | Zorbax RX-C8 (4.6 mm × 250 mm) |
| Solvents: | 50% CH₃CN, 50% 0.1% H₃PO₄ |
| Flow: | 1.0 mL/min |
| Sample volume: | 10 μL |
| Wavelength: | 210 nm |
| Retention times: | |
| NSA isomer | 2.7 min |
| 2-NSA 17 | 3.1 min |
| Toluene | 10.4 min |
| Naphthalene | 13.5 min |
| Sulfone impurity #1 | 25.5 min |
| Sulfone impurity #2 | 29.2 min |

Run 2—

Crude 2-NSA 17 (40 g; from Rutgers Organic Corp.; 88 A % and 76.6 wt. % pure) was mixed with 80 mL of acetonitrile and 320 mL of toluene. The mixture was heated to about 80-82° C. to dissolve all of the solid. The mixture was maintained at temperature and allowed to settle and form two layers. The bottom black layer (13.3 g containing about 13.6% of the acid) was cut. Water (2 mL) was added to the top layer, the mixture agitated and then allowed to cool to room temperature resulting in the formation of a slurry which was aged at room temperature overnight. The slurry was filtered and rinsed with toluene (50 mL) to afford a gray solid, which was vacuum dried at 60° C. to give 27.55 g of solid (98.1 A % and 90.0 wt. % pure). Recovery was 80.8%. 6.5% of the acid was lost in the mother liquor.

Run 3—

Crude 2-NSA 17 (40 g; from Rutgers Organic Corp.; 88 A % and 76.6 wt. % pure) was mixed with 80 mL of acetonitrile and 240 mL of toluene. The mixture was heated to about 80-82° C. to dissolve all of the solid. The mixture was maintained at temperature and allowed to settle and form two layers. The bottom black layer (13.73 g containing about 14.6% of the acid) was cut. Water (30 mL) was added to the top layer, the mixture agitated and then allowed to cool to room temperature and to settle which resulted in the formation of 2 layers. The top layer most of the organic impurities was cut (0.4 wt. % of the acid was lost). The bottom layer was concentrated to about 60 mL by vacuum distillation at less than 50° C. Acetonitrile (570 mL) was then slowly added to remove the water by continuous distillation. The final volume was about 60 mL. Tolume (20 mL) was added and the mixture heated to 60° C. providing a clear solution, which was then cooled to 45° C. and seeded with 2-NSA seed crystals which resulted in the formation of a slurry which was cooled to about 0-5° C. and aged for 30 minutes. The slurry was then filtered and rinsed with toluene (30 mL) to afford an off-white solid. After vacuum drying at 60° C., a solid acid was obtained (20.9 g, HPLC: 99.5 A % and 96.7 wt. % pure). Recovery was 66%. 21.5% of the acid was lost in the mother liquor.

Part B—Preparation of the Tris-NSA Salt of 16

| Material | MW (g/mol) | Amount | moles |
|---|---|---|---|
| Biarylpiperazine 16* | 427.42 | 2321 g | 5.43 |
| Seed of tris-salt 18 | 1052.14 | 12 g | |
| 2-Naphthalenesulfonic acid 17** | 208.24 | 3683 g | 16.29 |
| Acetonitrile | | 130 L | |
| Water | | 8.9 L | |

*15.3 wt % solution in CH₃CN, 94.8ee %
**92.1 wt % and 99.8 A %

2-Naphthalenesulfonic acid 17 (92.1 wt % pure) was dissolved in 21 L of CH₃CN and 8.82 L water at 65° C. A clear solution of biarylpiperazine 16 in CH₃CN (15.17 kg, 2.321 kg free base 6) was added over 1 min along with 1 L CH₃CN rinse. The mixture was still a clear solution (57° C). After seeding (12 g), a slurry formed gradually. The slurry was aged lh at 50-60° C. The slurry was vacuum distilled at 30-45° C. and 94 L CH₃CN was added slowly to reduce the water content in order to lower the solubility of the tris-NSA salt 18. Samples were taken during distillation to monitor the change:

| | Volume of | | Free base in | KE value of |
|---|---|---|---|---|
| Sample | CH₃CN added | ee % of salt | supernatant | supernatant |
| #1 | 72 L | 99.9% | 3.25 g/L | 6.8% |
| #2 | 84 L | 99.8% | 2.53 g/L | 4.6% |
| #3 | 94 L | 98.2% | 1.48 g/L | 3.5% |

The volume was adjusted to ˜49 L. The slurry was cooled to 25° C. and was aged overnight. The solids were filtered, rinsed with 12 L CH₃CN and dried in a vacuum oven at 60° C. to afford 5.29 kg crystalline solid 18 (99.5 A %, 41.2 wt %, 98.1 ee %, 94% recovery or 97% after ee % correction.) Loss in the mother liquor was 2.6%.

¹H NMR of 18 (DMSO-d6, with two drops of D2O, δ) 9.27 (t, 1H, J=6.3 Hz), 8.65 (d, 1H, J=1.6 Hz), 8.42 (d, 1H, J=1.7 Hz), 8.13 (d, 3H, J=0.8 Hz), 7.96˜7.94 (m, 3H), 7.91˜7.89 (m, 5H), 7.88˜7.85 (m, 3H), 7.72˜7.69 (m, 3H), 7.53˜7.51 (m, 6H), 4.03˜3.97 (m, 3H), 3.93 (s, 3H), 3.33˜3.24 (m, 2H), 3.02˜2.96 (m, 2H), 2.50˜2.45 (m, 2H), 1.56 (s, 6H).

| | |
|---|---|
| HPLC Assay: | |
| Column: | Zorbax RX-C8 (4.6 mm × 250 mm) |
| Solvents: | 60% CH₃CN, 40% 0.1% H₃PO₄ |
| Flow: | 1.0 mL/min |
| Sample volume: | 10 μL |
| Wavelength: | 210 nm |
| Retention times: | |
| Biarylpiperazine 16 | 2.1 min |
| 2-NSA 17 | 3.1 min |

Acetonide 21

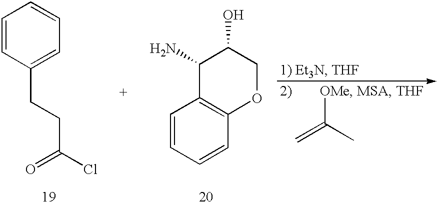

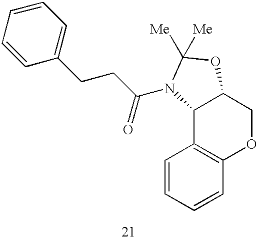

| Material | MW (g/mol) | Amount | | mmoles |
|---|---|---|---|---|
| Aminochromanol 20 | 165.19 | 100.00 | | 605 |
| Acetonide 21 | 337.41 | | | |
| Triethylamine (d = 0.726) | 101.19 | 98 | mL | 703 |
| Hydrocinnamoyl chloride* | 168.82 | 93 | mL | 622 |
| 2-Methoxypropene (d = 0.753) | 72.11 | 232 | mL | 2.42 |
| Methanesulfonic acid (d = 1.481) | 96.10 | 4.0 | mL | 62 |
| THF | | 2000 | mL | |
| IPAc | | 3000 | mL | |
| 5% Sodium bicarbonate | | 1800 | mL | |
| Cyclohexane | | 3850 | mL | |
| Water | | 900 | mL | |

*98%, d = 1.13

To a mixture of aminochromanol 20 (100.0 g, 95% ee, 605 mmol), TEA (89 mL, 635 mmol), and 1800 mL dry TKF at room temperature was added a solution of hydrocinnamoyl chloride (93 mL, 622 mmol, 1.03 eq) in THF (200 mL) over 40 min, allowing the temperature to drift up to 45° C. At the end of the addition, a slurry was generated which was aged at 45° C. for 30 min then cooled to 30° C. 2-Methoxypropene (232 mL, 4.0 eq) was added, followed by 4.0 mL methanesulfonic acid (0.10 eq). The mixture was aged at 35˜38° C. for 1 h. The flask was fitted with a condenser, and the slurry was warmed to 40° C., aged for 2 h, heated to 60° C. and aged at 60° C. under N₂ for 2˜4 h until BPLC showed <0.1 A % amide remaining. The reaction was quenched with 9 mL triethylamine. The mixture was concentrated to about 2 L by vacuum distillation at <60° C. IPAc (3 L) was added slowly to replace THF. The final volume was 2.4 L. The mixture was cooled to room temperature and 900 mL of 5% NaHCO₃ were added to dissolve all solids. After settling, the aqueous layer was cut and the organic layer was washed with 900 mL 5% NaHCO₃ and then 900 mL water. The organic layer was concentrated to 2.5 L by vacuum distillation at <85° C., and cyclohexane (3.6 L) was added slowly during distillation to solvent switch. Some solids formed during distillation. When the mixture was heated to 70-75° C., most of the solids dissolved. At the end of distillation all solid was dissolved by heating to 75˜80° C. The clear solution was cooled slowly to RT over 2.5 h during which slurry formed. This was aged 30 min at room temperature and 30 min at 0˜5° C. The slurry was filtered and the solids were rinsed with 250 mL cyclohexane. After vacuum oven drying at 50° C., 184.16 g (98.7 A %, 98.1 wt % pure) of acetonide 21 was obtained. There was 6.4% loss in the mother liquor. The yield after purity correction was 88%.

¹H NMR (CDCl₃, 300 MHz) 7.25 (m, 7H), 6.82 (m, 2H), 4.70 (d, 1H), 4.33 (m, 1H), 4.08 (d, 1H), 3.92 (s, 1H), 3.11 (m, 2H), 2.92 (m, 1H), 2.68 (m, 1H), 1.61 (s, 3H), 1.23 (s, 3H).

| | |
|---|---|
| HPLC Assay: | |
| Column: | Zorbax RX-C8 (4.6 mm × 250 mm) |
| Solvents: | 60% CH₃CN, 40% 0.1% H₃PO₄ |
| Flow: | 1.0 mL/min |
| Sample volume: | 10 μL |
| Wavelength: | 210 nm |
| Retention times: | |
| Aminochromanol 20 | 2.1 min |
| Hydroxyamide | 3.9 min |
| IPAc | 4.1 min |
| Acetonide 21 | 7.7 min |
| Ester impurity | 11.1 min |

### EXAMPLE 12

### Olefin 22

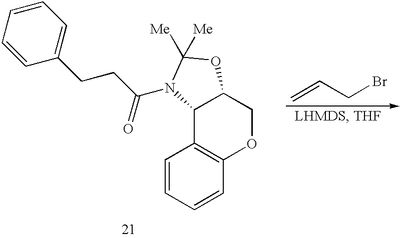

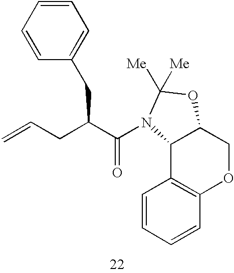

| Material | MW (g/mol) | Amount | | mmoles |
|---|---|---|---|---|
| Acetonide 21 (99.6 wt. %) | 337.41 | 50.00 | g | 148 |
| Olefin 22 | 377.48 | | | |
| Allylbromide | 120.98 | 18.60 | g | 154 |
| 1.38 M LHMDS in THF(d = 0.89) | 109 | g | 169 | |
| Citric acid | 192.13 g/mol | 8.63 | g | 44.9 |
| Tetrahydrofuran | | 343 | mL | |
| Isopropyl acetate | | 1100 | mL | |
| 0.3 M Sulfuric acid | | 180 | mL | |
| 5% Sodium bicarbonate | | 180 | mL | |
| Water | | 180 | mL | |

Acetonide 21 (50.00 g, 148 mol) was dissolved in 283 mL THIF (KF=116 μg/mL). The solution was degassed and was placed under N₂. The solution was cooled to −46 to −44° C. and 18.60 g (1.04 eq) allylbromide was added. LHMDS/THF (107 g) was charged over 45 min at −46 to −44° C. After a 60 min age at this temperature, a sample was taken (quenched into 2 vol cold IPA) for HPLC assay, which showed 0.68 A % acetonide 21 remaining (99.3% conversion). More LHMDS/THF (2.14 g) was added, and the mixture was aged for 30 min more. HPLC showed 0.22 A % acetonide 21 (99.8% conversion). The reaction was quenched by adding cold citric acid solution in THF (8.63 g/60 mL THF). A slurry formed. The slurry was warmed from −32° C. to 16° C. over 1 h. The batch was vacuum distilled to ˜400 mL at <40° C. and was flushed with 1100 mL IPAc to solvent switch to IPAc. The final volume was 450 mL. To the slurry was charged 180 mL 0.3 M H₂SO₄ (d=1.016 g/mL) at 20-25° C. All solids dissolved. After settling, the aqueous layer was cut and the organic layer was washed with 180 mL water and then 180 mL 5% NaHCO₃. The organic layer was diluted to 500 mL with IPAc. By HPLC the solution yield of olefin 22 was 98%. The concentration of olefin 22 was about 0.3 M. The solution was used in Example 13 without further purification.

¹H NMR (CDCl₃, 300 MHz) indicated a 5:1 mixture of rotamers: 7.30 (m, 5H), 7.05 (m, 1H), 6.80 (m, 1H), 6.4 (m, 1H), 5.85 (m, 1H), 5.15 (m, 1H), 4.98 (m, 1H), 4.40 (m, 1H), 4.25 (m, 2H), 3.38 (dd, 1H), 3.19 (m, 1H), 2.80 (m, 1H), 2.42 (m, 1H), 1.70 (s, 3H), 1.23 (s, 3H).

| | |
|---|---|
| HPLC Assay: | |
| Column: | Zorbax RX-C8 (4.6 mm × 250 mm) |
| Solvents: | 60% CH₃CN, 40% 0.1% H₃PO₄ |
| Flow: | 1.0 mL/min |
| Sample volume: | 10 μL |
| Wavelength: | 210 nm |
| Retention times: | |
| IPAc | 4.1 min |
| Allylbromide | 5.2 min |
| Acetone eliminated | 6.0 min |
| impurity | |
| Acetone adduct | 7.2 mm |
| Acetonide 21 | 7.7 mm |
| Olefin 22 | 12.6 min |
| Epi-olefin | 12.9 min |

### Example 13

### Iodohydrin 23

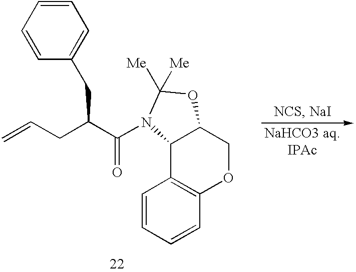

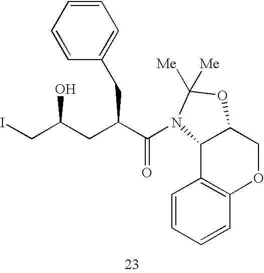

| Material | MW (g/mol) | Amount | | mmoles |
|---|---|---|---|---|
| Olefin 22 | 377.48 | | | ˜148 |
| Iodohydrin 23 | 521.39 | | | |
| NCS | 133.53 | 33.60 | g | 252 |
| 57% NaI | 149.89 | 64.22 | g | 244 |
| 20% Na thiosulfate pentahydrate | 248.18 | 165 | mL | |
| IPAc | | −50 | mL | |
| 5% Sodium bicarbonate | | 220 | mL | |
| Water | | 220 | mL | |

To the solution of olefin 22 (500 mL, ˜148 mmol) in IPAc was charged 220 mL water and 220 mL 5% NaHCO₃. The mixture was cooled to 3-4° C. NCS (33.60 g, 252 mmol, 1.7 eq) was added, then 57% Nal solution (64.22 g, 244 mmol, 1.65 eq) was added over 40 min at 4-7° C. The resulting brown solution was allowed to warm to 20° C. over 2 h and then was warmed to 30° C. over 15 min. The mixture was aged at 30° C. for 4 h. The conversion to iodohydrin was 98.6% after warming to 20° C. and 99.9% after 4 h age at 30° C. The batch was cooled to room temperature and then quenched with fast addition of 165 mL 20% Na₂S₂O₃.5H₂O (d=1.17 g/mL). After agitating for 2 min, the color of reaction mixture changed to orange from brown. The mixture was settled and the aqueous layer (650 mL) was cut. The organic layer (520 mL) was assayed and solution yield of iodohydrin was 83%. The solution was used in Example 14 without further purification.

¹H NMR (CDCl₃, 300 MHz) indicated a 5:2 mixture of rotamers: 7.30 (m, 5H), 7.05 (m, 1H), 6.82 (m, 1H), 6.60 (m, 1H), 5.92 (d, 0.3H), 5.58 (d, 0.7H), 4.45 (m, 2H), 4.20 (m, 2H), 3.63 (m, 1H), 3.44 (m, 2H), 3.20 (m, 2H), 2.82 (m, 2H), 2.40 (d, 1H), 2.00 (m, 1H), 1.72 (s, 3H), 1.49 (d, 2H), 1.29 (s, 3H).

| | |
|---|---|
| HPLC Assay: | |
| Column: | Zorbax RX-C8 (4.6 mm × 250 mm) |
| Solvents: | 60% CH₃CN, 40% 0.1% H₃PO₄ |
| Flow: | 1.0 ml/min |
| Sample volume: | 10 μL |
| Wavelength: | 210 nm |
| Retention times: | |
| IPAc | 4.1 min |
| Iodohydrin 23 | 9.3 min |
| Olefin 22 | 12.6 min |

### EXAMPLE 14

### Epoxide 24

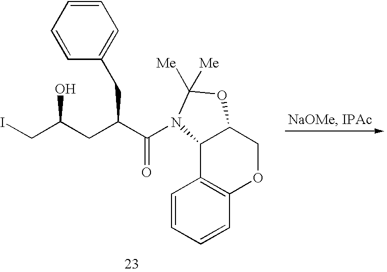

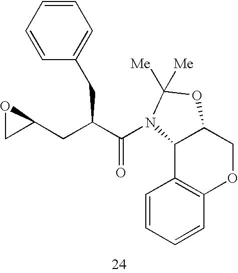

| Material | MW (g/mol) | Amount | | mmoles |
|---|---|---|---|---|
| Iodohydrin 23 | 521.39 | | | <148 |
| Epoxide 24 | 393.48 | | | |
| 25% NaOMe in MeOH | 54.02 | 44.8 | g | 207 |
| IPAc | | 500 | mL | |
| IPA | | 450 | mL | |
| 10% Sodium sulfate decahydrate | | 340 | mL | |
| Water | | 170 | mL | |

The solution of iodohydrin 23 in IPAc (520 mL, <148 mmol) was vacuum distilled at <35° C. IPAc (500 mL) was added slowly while the volume of solution was maintained at 500 mL. KF of the solution was <1400 μg/mL after the distillation. After azeotropic drying, the organic solution was cooled to 14-16° C. Then 44.8 g 25% NaOMe in methanol was added (small endotherm). The mixture was aged at 15° C. for 45 min. Sampling after 30 min age at 14-16° C. showed >99.7% conversion to epoxide. The reaction was quenched at 15-20° C. by adding 170 mL water. The mixture was agitated 2 min and settled 10 min. The aqueous layer was cut. The clear, dark brown organic layer was washed by 2×170 mL 10% Na₂SO₄—10H₂O (d=1.04 g/mL). The pH of the first wash aqueous solution was 7 and was 6.5 for the second wash. The loss of epoxide in these two washes was <0.1%. The organic layer showed a lower 99.3% conversion to epoxide, due to some reverse reaction to iodohydrin. The organic layer was vacuum distilled to 220 mL and then flushed with 400 mL IPA at <45° C. A slurry was generated during this solvent switch. The slurry was heated rapidly to 80° C. to dissolve all solid. The dark solution was cooled slowly to 60-65° C. and was aged at this temperature to obtain a thin slurry. The slurry was cooled to room temperature over 1 h and was cooled to 0˜5° C. for 3 h. The slurry was filtered and the cake was displacement-rinsed with 50 mL cold IPA. By HPLC there was 2.4% epoxide lost in mother liquor and rinse (160 mL). The cake was vacuum oven dried overnight at 40° C. with a nitrogen sweep to afford 48.02 g of epoxide 24 (99.4 A % and 98.5^{wt}% pure). The yield was 81% from acetonide 21.

¹ H NMR (CDCl₃, 300 MHz) indicated a 5:2 mixture of rotamers: 7.30 (m, 5H), 7.10 (mn, 1H), 6.82 (mn, 1H), 6.50 (m, 1H), 5.89 (d, 0.3H), 5.40 (d, 0.7H), 4.40 (m, 2H), 4.15 (mn, 2H), 3.40 (m, 2H), 3.00 (mn, 1H), 2.85 (m, 2H), 2.50 (dd, 0.7H), 2.40 (dd, 0.3H), 2.20 (m, 1H), 1.72 (s, 3H), 1.49 (d, 1H), 1.29 (s, 3H).

| | |
|---|---|
| HPLC Assay: | |
| Column: | Zorbax RX-C8 (4.6 mm × 250 mm) |
| Solvents: | 60% CH₃CN, 40% 0.1% H₃PO₄ |
| Flow: | 1.0 mL/min |
| Sample volume: | 10 μL |
| Wavelength: | 210 nm |
| Retention times: | |
| IPAc | 4.1 min |
| Epoxide 24 | 8.0 min |
| Iodohydrin 23 | 9.3 min |

### EXAMPLE 15

### Preparation of Compound A Penultimate 25

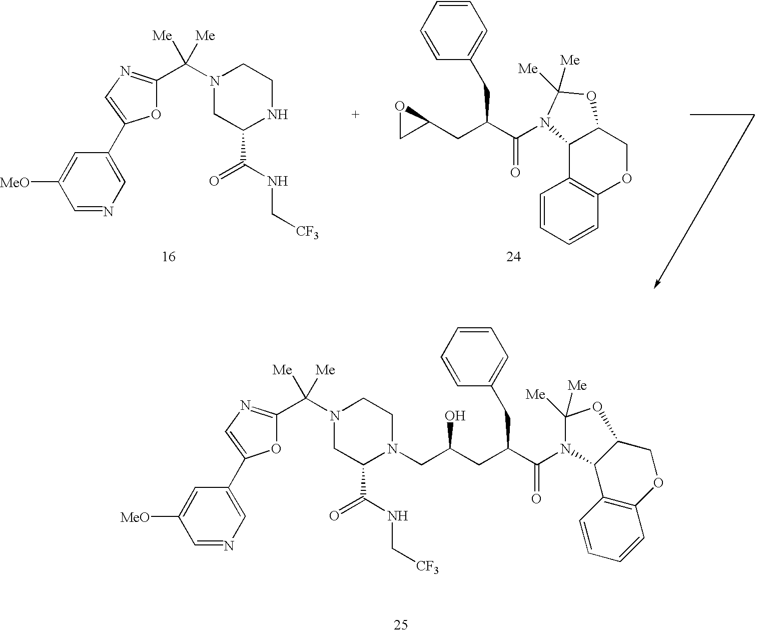

Biarylpiperazine tris-NSA salt (300.00 g, GMP) was slurried in MEOH (940 mL) and KOH in MeOH (860 mL, 1.0N). The slurry was allowed to stir for 4 h. MeOH was distilled off at 35 Torr with an internal temperature of 5° C. After ˜800 mL was distilled off, the slurry became too thick to stir and toluene (180 mL) was added. A total of 3600 mL of toluene was used to flush the slurry (mother liquors were checked for the presence of naphthalenesulfonic acid). The slurry was then filtered, rinsing 2×360 mL toluene. The filtrates were assayed by HPLC and found to contain 123.1 g biarylpiperazine. The filtrate was then concentrated and diluted with 480 mL t-amyl alcohol. It was concentrated again and then flushed with 450 mL t-amyl alcohol. It was assayed and found to contain 115.1 g biarylpiperazine. Epoxide 24 (107.00 g, 1.01 eq.) was added, and the mixture was stirred at 55° C. (internal temperature) for 90 h. The mixture was diluted with IPAc (1720 mL) and assayed for the coupled acetonide product 25 by BPLC (found 185.00 g (84% yield). Silica gel (370.0 g) and Darco G-60 activated carbon (46.25 g) were added and the mixture was heated at 50° C. for 1 hour. It was filtered through Solka Floc and rinsed with 925 mL 5% MeOH/IPAc (4×). The initial filtrate and first rinse were assayed and were found to contain a total of 146.03 g. Rinses 3 and 4 contained 24.69 g and 7.52 g, respectively. The filtrate, first and second rinses were combined. A portion of this containing ˜100 g was chromatographed (16 cm column, 2.00 kg silica) using 0 to 6% MeOH/IPAc. Clean fractions were combined and concentrated.

### EXAMPLE 16

### Preparation of Compound A 26

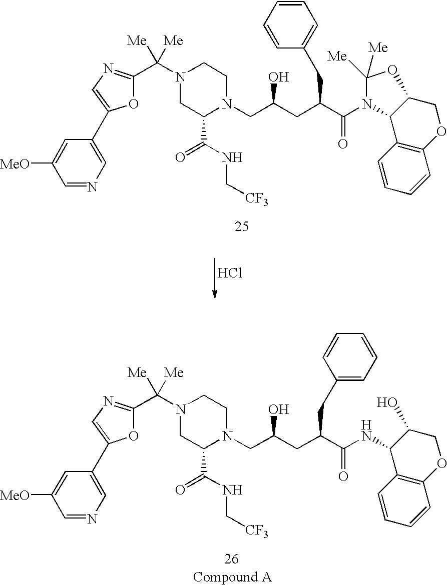

Compound A penultimate prepared in Example 15 (97.5 g) was dissolved in 225 mL MeOH and cooled to −10° C. 5.02N HCl in methanol (245 mL) was added dropwise over 30 min, keeping the temperature below 0° C. It was then transferred to a 0° C. bath. After stirring for 13 h, it was assayed and found to be greater than 98.5% complete. 5N NaOH (250 mL) was added, keeping the temperature below 0 C. After addition was complete the pH was checked and found to be 9. IPAc (1.0 L) and water (200 mL) were added and the layers were shaken to dissolve a brown oil that formed during the quench. The layers were cut and the aqueous layer was assayed and found to contain 0.19 g of Compound A. The organic layer was washed with 200 mL brine. The organic layer was assayed and found to contain 85.95 g of Compound A free base (92.7%). The brine layer was found to contain 0.05 g of Compound A. Activated carbon (17.99 g) was added and the mixture was stirred at 50° C. for 1 h. After cooling to room temperature the slurry was filtered through solka-floc and the cake washed with IPAc, 3×180 mL. The filtrate and washes were combined and assayed which showed 80.54 g of Compound A free base. The combined filtrate and washes were then concentrated to a yellow foamy solid.

¹H NMR (CD₃OD, 500 Hz): δ8.49 (s, 1 H), 8.22 (d, J=1.6Hz, 1 H), 7.66-7.67 (m, 1 H), 7.20-7.25 (m, 4 H), 7.14-7.17 (m, 1 H), 7.06-7.10 (m, 2 H), 6.80 (t, J=7.6 Hz, 1 H), 6.71 (d, J=8.0 Hz, 1 H), 5.13 (d, J=3.8 Hz, 1 H), 4.04-4.06 (m, 2 H), 3.92-3.98 (m, 1 H), 3.94 (s, 3 H), 3.78-3.82 (m, 1 H), 3.72-3.77 (m, 2 H), 3.06-3.10 (m, 1 H), 2.96-3.03 (m, 2 H), 2.88-2.94 (m, 1H), 2.85 (d, J=11.2 Hz, 1 H), 2.70-2.77 (m, 2 H), 2.63-2.67 (m, 1 H), 2.44-2.50 (m, 1 H), 2.34-2.44 (m, 4 H), 2.00-2.04 (m, 1 H), 1.60 (s, 3 H), 1.59 (s, 3 H), 1.35-1.38 (m, 1 H). LC-MS (M⁺+1) (EI) 781.5.

### EXAMPLE 17

### Crystalline Hydrochloride Salt of Compound A—Form I

Compound A free base (80.54 g) was dissolved in 408 mL of IPAc and then further diluted with 203 mL of IPA and heated to 60 C. HCl in EPA (50.4 mL, 0.25 eq., 0.518N, from conc. HCl) was added followed by seed crystal (81 mg Compound A-HCl as a slurry in 2 mL of 1:1 IPAc:IPA). The slurry was aged for 60 minutes and then an additional 151 mL of HCl in EPA (0.75 eq, 0.518N) added over 30 minutes. The resulting slurry was then aged for 2 hrs at 60 C. then allowed to cool to RT overnight. LC assay showed a loss of 8.4 mg/mL to the mother liquors prior to filtration. The slurry was filtered and the cake washed with 1:1 IPAc:IPA, 2×150 ml. After drying (40 C., 23 in Hg, N₂ sweep) a total of 72.34 g of crystalline Compound A-HCl was obtained.

¹H NMR (CD₃OD, 400 MHz): δ=8.50 (d, J=1.5 Hz, 1H), 8.25 (d, J=2.7 Hz, 1H), 7.70 (s, 2H), 7.32-7.17 (m, 5H), 7.16 (d, J=7.6 Hz, 1H), 7.09 (m, 1H), 6.86 (m, 1H), 6.40 (d, 8.1 Hz, 1H), 5.17 (d, J=4.0 Hz, 1H), 4.84 (s, 4H), 4.21 (bs, 1H), 4.17-3.98 (m, 4H), 3.96 (s, 3H), 3.97-3.75 (m, 3H), 3.40 (bs, 1H), 3.32-3.13 (m, 2H), 3.07 (bs, 1H), 3.03-2.93 (m, 2H), 2.85-2.62 (m, 3H), 1.90 (m, 1H), 1.68 (s, 6H), 1.53 (m, 1H).

Co-injection of the isolated salt with Compound A free base showed a single peak on HPLC (column: YMC basic, 25 cm×4.6 mm; solvent: mixture of acetonitrile and 0.1% H₃PO₄ in water).

The HCl salt was analyzed by differential scanning calorimetry at a heating rate of 10° C./min in a closed cup under flowing nitrogen and was found to have a DSC curve exhibiting an endotherm with an extrapolated onset temperature of about 205° C., a peak temperature of about 207° C. and an associated heat of about 132 J/gm. Based on results of TG and TG-FTIR, the endotherm is due to crystal melting.

The XRPD pattern was generated on a Philips Analytical X-ray instrument with XRG 3100 control and APD 3720 control. Copper K-Alpha 1 radiation was used as the source. The experiments were run under ambient conditions. The XRPD pattern was found to have characteristic diffraction peaks corresponding to d-spacings of 3.1, 3.5, 3.8, 3.9, 4.2, 4.3, 4.9, 5.7, 6.6 and 15.7 angstroms.

### EXAMPLE 18

### Crystalline Hydrochloride Salt of Compound A—Form II

Form I crystalline Compound A.HCl salt (100 mg) was added to about 30 mL of hot acetonitrile (heated to boiling). Additional portions of hot acetonitrile were added to provide a total amount of hot acetonitrile sufficient to just dissolve the HCl salt. The solution was then cooled to 5° C., and the resulting solid was recovered by filtration and air dried overnight. The recovered solids were characterized by DSC, XRPD, and TGA. The salt was analyzed by DSC at a heating rate of 10° C. per minute in a closed cup under flowing nitrogen and was found to have a DSC curve exhibiting an endotherm with an extrapolated onset temperature of about 162.1° C., a peak temperature of about 174.8° C. with a corresponding heat of fusion of about 48.6 J/g. TGA analysis showed a 0.15% weight loss between 25° C. and 125° C. indicating no volatiles and ruling out the possibility of a solvate.

The XRPD pattern was generated on a Siemens D5000 X-ray diffractometer (ma=40; kV=50 with copper K-alpha 1 radiation). The experiments were run under ambient conditions. The XRPD pattern was found to have characteristic diffraction peaks corresponding to d-spacings of 3.7, 3.9, 4.1, 4.3, 4.5, 4.6, 4.8, 5.0, 5.3, 5.6, 6.2, 6.4, 6.7, 10.6, and 15.7 angstroms. The d-spacings for the most intense peaks in the XRPD pattern were 3.7, 3.9, 4.5, 4.6, 5.0, 5.6, 6.2, 6.4, 6.7 and 15.7 angstroms.

### EXAMPLE 19

### Crystalline Hydrobromide Salt of Compound A

Compound A free base (0.040 g) was dissolved in 1 mL of IPAc. HBr (0.350 mL of a 0.147 M solution in EPA) was added dropwise to the IPAc solution of Compound A. A precipitate formed initially, but dissolved with stirring. The solution was seeded with crystalline Compound A HBr salt and left overnight. The resulting slurry was filtered to give 0.027 of a crystalline HBr salt of Compound A.

¹H NMR (CD₃OD, 400 MHz) δ=8.48 (d, J=1.5 Hz, 1H), 8.23 (d, J=2.7 Hz, 1H), 7.67 (s, 2H), 7.27-7.15 (m, 5H), 7.12 (d, J=7.6 Hz, 1H), 7.07 (m, 1H), 6.84 (td, J=7.5, 1.1 Hz, 1H), 6.71 (dd, J=8.2 Hz, 1.1 Hz, 1H), 5.15 (d, J=4.1 Hz, 1H), 4.80 (s, 8H), 4.18 (bs, 1H) 4.11-3.95 (mn, 4H), 3.40 (bs, 1H), 3.29-3.10 (4H), 3.04 (bs, 2H) 2.98-2.90 (m, 2H) 2.78-2.60 (mn, 3H), 1.86 (m, 1H), 1.64 (s, 6H), 1.50 (mn, 1H)

The isolated material was observed to be birefringent under the microscope.

Co-injection of the isolated salt with Compound A free base showed a single peak on HPLC (column: YMC basic, 25 cm×4.6 mm; solvent: mixture of acetonitrile and 0.1% H₃PO₄ in water).

The HBr salt was analyzed by differential scanning calorimetry at a heating rate of 10° C./min in a closed cup under flowing nitrogen and was found to have a DSC curve exhibiting an endotherm with an extrapolated onset temperature of about 193° C., a peak temperature of about 204° C. and an associated heat of about 78 J/gm. Based on results of TG and TG-FTIR, the endotherm is due to crystal melting.

The XRPD pattern was generated on a Philips Analytical X-ray instrument with XRG 3100 control and APD 3720 control. Copper K-Alpha 1 radiation was used as the source. The experiments were run under ambient conditions. The XRPD pattern was found to have characteristic diffraction peaks corresponding to d-spacings of 3.1, 3.8, 3.9, 4.2, 4.8, 5.6, 6.7, 10.4 and 15.8 angstroms.

### EXAMPLE 20

### Formulation for Oral Administration

As a specific embodiment of an oral composition, 100 mg of the compound of Example 17 is formulated with sufficient finely divided lactose to provide a total amount of 580 to 590 mg to fill a size O hard gel capsule.

While the foregoing specification teaches the principles of the present invention, with examples provided for the purpose of illustration, the practice of the invention encompasses all of the usual variations, adaptations and/or modifications that come within the scope of the following claims.

What is claimed is:

1. A hydrohalide salt of Compound A, wherein Compound A is of formula: 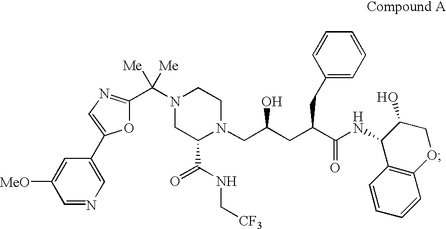
and the hydrohalide is hydrochloride or hydrobromide.

2. The hydrohalide salt according to claim 1, which is a hydrochloride salt of Compound A.

3. The hydrohalide salt according to claim 1, which is a hydrobromide salt of Compound A.

4. The hydrohalide salt according to claim 1, which is a crystalline salt of Compound A.

5. A Form I crystalline hydrochloride salt of Compound A, characterized by crystallographic d-spacings of 4.2, 4.9, and 5.7 angstroms; wherein Compound A is of formula:

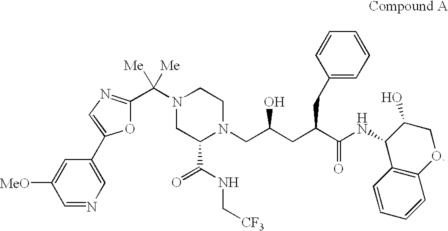

6. The Form I crystalline hydrochloride salt of Compound A according to claim 5, which is characterized by crystallographic d-spacings of 3.8, 3.9, 4.2, 4.3, 4.9, and 5.7 angstroms.

7. The Form I crystalline hydrochloride salt of Compound A according to claim 5, which is characterized by crystallographic d-spacings of 3.1, 3.5, 3.8, 3.9, 4.2, 4.3, 4.9, 5.7, 6.6 and 15.7 angstroms.

8. The Form I crystalline hydrochloride salt according to any one of claims 5 to 7, which is further characterized by a differential scanning calorimetry curve, at a heating rate of 10° C./min in a closed cup under flowing nitrogen, exhibiting an endotherm with a peak temperature of about 207° C. and an associated heat of about 132 J/gm.

9. A Form II crystalline hydrochloride salt of Compound A, characterized by crystallographic d-spacings of 5.0, 6.4, and 15.7 angstroms; wherein Compound A is of formula:

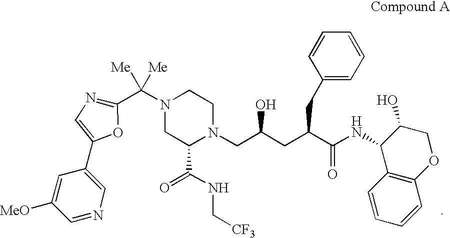

10. The Form II crystalline hydrochloride salt of Compound A according to claim 9, which is characterized by crystallographic d-spacings of 4.5, 5.0, 5.6, 6.2, 6.4, and 15.7 angstroms.

11. The Form II crystalline hydrochloride salt of Compound A according to claim 10, which is characterized by crystallographic d-spacings of 3.7, 3.9, 4.5, 4.6, 5.0, 5.6, 6.2, 6.4, 6.7 and 15.7 angstroms.

12. The Form II crystalline hydrochloride salt according to any one of claims 9 to 11, which is further characterized by a differential scanning calorimetry curve, at a heating rate of 10° C./min in a closed cup under flowing nitrogen, exhibiting an endotherm with a peak temperature of about 174.8° C. and an associated heat of fusion of about 48.6 J/gm.

13. A crystalline hydrobromide salt of Compound A, characterized by crystallographic d-spacings of 3.8, 3.9, and 4.2 angstroms; wherein Compound A is of formula:

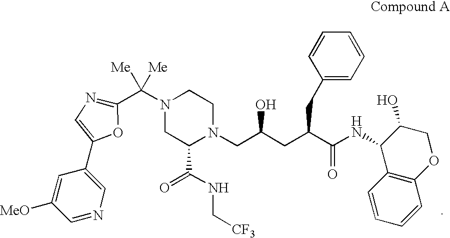

14. A crystalline hydrobromide salt of Compound A according to claim 13, characterized by crystallographic d-spacings of 3.1, 3.8, 3.9, 4.2, 4.8, and 5.6 angstroms.

15. A crystalline hydrobromide salt of Compound A according to claim 13, characterized by crystallographic d-spacings of 3.1, 3.8, 3.9, 4.2, 4.8, 5.6, 6.7, 10.4 and 15.8 angstroms.

16. The crystalline hydrobromide salt according to any one of claims 13 to 15, which is further characterized by a differential scanning calorimetry curve, at a heating rate of 10° C./min in a closed cup under flowing nitrogen, exhibiting an endotherm with a peak temperature of about 204° C. and an associated heat of about 78 J/gm.

17. A pharmaceutical composition comprising a therapeutically effective amount of a salt of Compound A as recited in any one of claims 1 to 16 and a pharmaceutically acceptable carrier.

18. A pharmaceutical composition made by combining a therapeutically effective amount of a salt of Compound A as recited in any one of claims 1 to 16 and a pharmaceutically acceptable carrier.

19. A method of preventing or treating HIV infection, delaying the onset of AIDS, or treating AIDS, which comprises administering to a subject in need thereof a therapeutically effective amount of a salt of Compound A as recited in claim 1.

20. A method of inhibiting HIV protease, which comprises administering to a subject in need of such inhibition a therapeutically effective amount of a salt of Compound A as recited in claim 1.
